SEQUENCE LISTING (14 sequences)

SEQ ID NO: 1 - protein, 30 aa, Artificial
  Synthetic
RKKRRXRRRASNRELVVDFLSYKLSQKGYS

SEQ ID NO: 2 - protein, 30 aa, Artificial
  Synthetic
RKKRRQRRRASNRELVVDFLSYKLSQKGYS

SEQ ID NO: 3 - protein, 30 aa, Artificial
  Synthetic
RKKRRXRRRASNRELVVDFLSDKLSQKGDS

SEQ ID NO: 4 - protein, 30 aa, Artificial; identical to SEQ ID NO: 3
  Synthetic

SEQ ID NO: 5 - protein, 31 aa, Artificial
  Synthetic
CRKKRRXRRRASNRELVVDFLSYKLSQKGYS

SEQ ID NO: 6 - DNA, 23 bp, Mus musculus
ggguguuugcaaaugauuauutt

SEQ ID NO: 7 - DNA, 23 bp, Mus musculus
  misc_feature (1)..(1): Phosphorylated
uaaucauuugcaaacacccuutt

SEQ ID NO: 8 - DNA, 4919 bp, Homo sapiens
acttcgctccgcgcagccgcctggtctgcagtttgttggagctctgcgtccagcgccgctgccgctgccgccgccgccgccgccgccgccgccgccgccgccgccgccactaccaccacttgattcttgcagccaccctgcgaaccctgccacactgcgatcgcatcatcgcggtattcggttcgctgcgttcccgccgccaccgcctcggcgccctttcttggcccttgttcccccaaatgtctgactctgactctcggactgagaaacgcaagaaaaaaagaccaaatggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattgacaggttctttgcggagccgagataccatgcagacattttgcttgttcaaaccaacaagacccagcaccgcggtctcctggtgccattattatgcagccagcggttctcttgtggagggggcaggtgacgtttcagaagacaccgagctggatgggactacctttctgttcatcaccacacagcagaatttctaatggaagtttgttgtgaatgtaaaggagggagcattctttgctttttaatatacaaaccatggttttttggagcaggattttgtgtaagaatggtgtttacatgcagtgtgttttccccctcaccttcaataaggtttttcaaaaaggaaatggaaactttttaaccaatttgtgaataacttttgtattaaaattttaagaacctacggcctattctcagaggattatgtaacccctgcagtggaaactgagccagctaacttaaaaagctgccttagtttatttttagagattacagaatttttaaacagggagacgtgtgatatactccctcccttccctactattgcctctctgacctttttaaattatttttaataccaaaagagttcttttgaaatggaactgattaaaagggcagagggtctgttgccagcctgcattgatataccagtcccatttgtaaatatttacgtacctttataaattcagttgcatctgtggcaaaatttcagactatttttgcgtctttcctcatcactttttgtgatgcaactccagtctggactcagatgcatagatttggtccagtgtattttcatgataaagtgaaattgagtcagaacaagagttaatatctgcctgtatcttgcacagttcgagcgatctgttattaactgggaagcatttggtgttggttttcattccatttcgacgagcatgttattgggaagtattctgaagaggcaatagcagtaataacaacagacttaagtgctacgcccctttgtgctgctggcttttctggttgcaggctttcccatggtcacaggatgcactgtcagcatcaggtcccagagggccaccgtgtccattacagcagagtccagctgcagcatccagctcacgccctcatgggaattggcacaggcctggggcagggcttctgatggccatttgcttggcctcctgcattttagtccaactcacagtccactagcttcactcctttaaattcactttgaaacaggcctcatcccacttccaccagcaccatagaagaataattctgggcagaagtctgttttttttcatttttccaggacagttggatattgtcaggccacttgtgaccccagccatgtagtgagggtgctctttctctgtgcctgctccttatgagtgcagtggaaggaagccacacactggtcagtcatttcagaggcagcagatgcccagggagacccaagaaagagtcaggttagggagcagtgaaagtgaggagggaagacaattctgtgaactctgtaactcttaaaatttttgaaaactccatcgttaaacaacttttaaaagaaataactaaattttcaaatgagtaagcagtgccaccaactagtgttttgcccgatagaagagccagcatgttcacgttatttaaattaggtggaaaaatctaaacatttttatcttcataatttaaaaaatatatatgtatatattgcatattcactttttcctttaggtagagatgatttcaatccaaatactcttactttaaaaaatttcctttccccaagaatctccttgggactttgacttatttttaaagctgtgttggagctcatcttgttccctgatgtgtctcgagcccattggtagggtcatacaaagcccacggttacaagcagtggtaggattgcagccgtgggcctgctggacacacacatacaccaaagatgtatttggatctgggcaccccctcccaggatccctgtactcacgtgccagtctcctgactagagcactttactctgtttcctcagccctgcagcccctgggagcacacactgggtgcagccctgggccaggcacgggaggccctgccctgtgctgcccaggggctgtgtgcaccacatgagcacatttccctctggcctggcggcctccaggctggctgtggaaacagttcctgaggaaattagagattctatgaattgtaggagtattaaagaccaggctgttggcaccagaacttaaagcgatgactggatgtctctgtactgtatgtatctggttatcaagatgcctctgtgcagaaagtatgcctcccgtgggtatacgtttttaccttttttaaaaaacatttttgtagaaaaaataattaaatcccctttttggaaacttactgcaggttttgtgccttgacaacctctccctatgtgaggtttgtaaaaagtgtcctgtgacttaacacagaaacgcaataaacacacacaaaatagtttcatgagtgattcttcagatgcccttcccaactggttagttgatcaagaattttgggggtgggggttgcggagaaatcaagtttaaaattccttctgattaaaaaaatatagtggaatacaattgtctgccgtttccccttcttaatgtatatattgtgagtatttattagattcgtaggtcatattacttatcaactgagccaaatgtctgtgtgcaattgtgtttcctttaccttgtaaaattttgtacagcataaataagtaaaaaaatcactgtttttctcaactttttcaaaatcaaggattgtaaatattgtagattctttttctgtgtgatgtgtcctactgtttcataatgctgtaacttgtagaaatattgtatatttattttctgcttatttaatgtcttaatttctgaaaagtattaacatccctgtctcccactcccctgccgtcccatgaagttaactcctgagagttgtcgggggtgactggagagctcattgcagaccacgtggtcctccagggtggctctccaccttcgggtcctggtatttccagtcaagtgggtttcaattcttgggctttgccgcccttatgatgaagtgtgtgtttgatgccagtgagaaactcagtctggcaggctacaaaattctactccaagaaatacccagcaaccttctgtttgttccaaagcaactagcttatcatgcaagcaaattttgctgactccaggctttatctttaggaaaacaaaaaaaccaaagtattatcagcaggtgggaaagatttttctattgaaaatttatccctgacaactcagcgtttagaaaagaaataaaatgtgccacttccagaggtgctgcattgcagttgttcagggctagggccaggcaggacaagtgaatgggtgggacaggtggctcctgcctaaggaccacctcaggccactaaccccttgtggacaactgtgagtagctgggttttcccccacctgctgtgcaacttcctgtgctttgaggttggactaacttgtcttcaggagctaattaactgtacagccctccccacgccccacccatacggtcactgcatttggtcagcctgcttcttcaggtcgatgccctccttctgatactccatctccttcaggggaggttggggccccactggactgggtgtcaagatgtgaaagcttatgggagctttaaggagacttcatggtggttccatgcaggtggttctgccatccctgctgatttagcctggtgcctgtgtgtgtccactcacgtacacgtggggtgggggaaacgtgtctacagatgacgctaaatcagttggggtctactctaaacagcattgtgtgtaagaagcatcctcaagctcccagttaagtaacttgactacttttatttgggaatttcagactatagaagctctcttatgttttatgtccagattctgtgaccactagttactgtatcagaactcatcaggtacccacttataaatagcactgatctggctgtatactgatccatcactaacctgttttctaggacccagcgtatgtagcatttgtattgcagtttccctggcttacttgtgttttgcactgatgaattttgacagggtaattgccactttacttgtgcaatactgctgtaaataactgcagatttttaaacaatcttttatgttaattttataaaaataaaactttcaactagtt

SEQ ID NO: 9 - DNA, 23 bp, Homo sapiens
ucuuacgacuguuacguuauutt

SEQ ID NO: 10 - DNA, 23 bp, Homo sapiens
  misc_feature (1)..(1): Phosphorylated
uaacguaacagucguaagauutt

SEQ ID NO: 11 - DNA, 23 bp, Artificial
  Synthetic
ggcaagcacccugaaguucuutt

SEQ ID NO: 12 - DNA, 25 bp, Artificial
  Synthetic
gaacuucagggucagcuugccuutt

SEQ ID NO: 13 - RNA, 21 nt, Mus musculus
cgacacagaguuccagaauuu

SEQ ID NO: 14 - RNA, 21 nt, Mus musculus
gagaugaacuggacagcaauu

(19) United States
(12) Patent Application Publication
(10) Pub. No.: US 2009/0012025 A1
(43) Pub. Date: Jan. 8, 2009

(54) Methods and Compositions for Treatment of Sepsis

(76) Inventors: Richard Hotchkiss, Chesterfield, MO (US); Jonathan McDunn, University City, MO (US); David Piwnica-Worms, St. Louis, MO (US); Steven Schwulst, St. Louis, MO (US)

Correspondence Address: WASHINGTON UNIVERSITY-SNR;C/O SONNENSCHEIN NATH & ROSENTHAL L.L.P, P.O. BOX 061080, WACKER DRIVE STATION , SEARS TOWER, CHICAGO, IL, 60606, US

(21) Appl. No.: 12/029,412

(22) Filed: Feb. 11, 2008

Related U.S. Application Data
(63) Continuation-in-part of application No. 11/391,964, filed on Mar. 29, 2006.
Application No. 11/391,964 is a continuation-in-part of application No. 11/286,920, filed on Nov. 23, 2005.
(60) Provisional application No. 60/921,492, filed on Feb. 9, 2007.

(51) Int. Cl. A61K 31/7105; A61P 31/04

(52) U.S. Cl. 514/44

(57) ABSTRACT

Methods of treatment of sepsis are disclosed. These methods comprise administering to a subject a composition comprising at least one siRNA directed against at least one gene encoding a pro-apoptotic polypeptide. The pro-apoptotic polypeptide, in some aspects, can be other than Fas or caspase-8. In some embodiments, an siRNA can be directed against a pro-apoptotic component of the mitochondrial pathway, such as a pro-apoptotic bcl-2 protein. In some aspects, an siRNA can be directed against a BH3-only bcl-2 protein, while in other aspects, siRNAs can be directed against multi BH domain Bcl-2 family members such as bax and bak. In some embodiments, an siRNA can be directed against a death receptor pathway molecule such as FADD. In various configurations, a composition can also comprise a cationic lipid such as DOTAP, or nanoparticles comprising a cyclodextrin-containing polycation and a polymer such as a poly(ethylene glycol).

### RELATED U.S. PATENT APPLICATIONS

This application claims priority to U.S. Provisional application 60/921,492 filed Feb. 9, 2007. This application is also a continuation-in-part of application Ser. No. 11/391,964 entitled Membrane-Permeant Peptides for the Treatment of Sepsis filed Mar. 29, 2006, which is a continuation-in-part of application Ser. No. 11/286,920 entitled Membrane-Permeant Peptide Complexes for treatment of sepsis, filed Nov. 23, 2005. The present application claims benefit of these applications, which are herein incorporated by reference in their entirety.

### INTRODUCTION

1. Field

The present invention broadly relates to the field of medicine, in particular the field of pharmaceutical therapy. The present teachings disclose methods and compositions for the treatment of particular disorders, including sepsis.

2. Description

Sepsis

Sepsis is a major and growing health problem. Deaths due to sepsis and the often resulting organ failure are approaching a quarter million patients per year in the United States alone. Postmortem examinations of sepsis victims have revealed new insights into the pathophysiology of sepsis. For example, it is now known that patients who die of sepsis demonstrate profound depletion of T and B lymphocytes. (See, e.g., Crit. Care Med. 2005 July; 33(7): 1538-48). However, sepsis remains a difficult condition to treat because of the speed with which it develops and the lack of treatment options that can rapidly deliver systemically effective treatment. Despite advances, therapeutic approaches to the treatment of sepsis have remained limited.

Apoptosis

Apoptosis is a major form of programmed cell death (PCD) which occurs in multi-cellular organisms. Apoptosis involves a series of biochemical events leading to a characteristic cell morphology and death. There are two major cellular pathways for inducing apoptosis. One route involves the mitochondria (the “intrinsic,” “Bcl-2-regulated” or “mitochondrial” pathway), while the second route involves activation of death receptors (the “extrinsic,” or “death receptor” pathway). Both pathways can lead to activation of “executioner” proteins such as caspases.

The mitochondrial pathway involves pro-apoptotic and anti-apoptotic proteins of the bcl-2 family. Pro-apoptotic bcl-2 proteins are often found in the cytosol, where they act as sensors of cellular damage or stress. Following cellular stress, they relocate to the surface of the mitochondria where they can interact with anti-apoptotic proteins. Interactions between pro- and anti-apoptotic proteins can disrupt the normal function of the anti-apoptotic bcl-2 proteins, and can lead to the formation of pores in the mitochondria as well as release of cytochrome C and other pro-apoptotic molecules from the inter-membrane space. Formation of the apoptosome and the activation of the caspase cascade can then ensue. In the mitochondrial pathway, a myriad of diverse stress stimuli cause activation of “BH3-only” pro-apoptotic members of the Bcl-2 family that include, for example, Bad, Bik, Bid, Puma, Bim, Bmf, Noxa and Hrk (see, e.g., Bouillet, P., et al., Science 286: 1735-1738, 1999; Wang, K., et al., Genes and Development 10: 2859-2869, 1996, Fadeel, B., et al., FASEB J. 13: 1647-1657, 1999). These BH3-only proteins bind to and neutralize the anti-apoptotic members of the Bcl-2 family, such as Bcl-2, Bcl-xL, and Mcl-1, thereby unleashing the pro-apoptotic multi BH domain Bcl-2 family members Bax and Bak (Sedlak, T. W., et al., Proc. Nat'l. Acad. Sci. USA 92: 7834-7838, 1995; Chittenden, T., et al., Nature 374: 733-736, 1995; Oltvai., Z. N., et al., Cell 74: 609-619, 1993; Tzung, S. P., et al., Am. J. Pathol. 150: 1985-1995, 1997).

In contrast, the death receptor pathway involves activation of members of the tumor necrosis factor (TNF) receptor (TNF-R) superfamily comprising an intra-cellular death domain, including Fas, TNF-R1, DR3, and receptors for TRAIL (Pan, G., et al. Science 276: 111-113, 1997; Yeli, W.-C., et al., Science 279: 1954-1958, 1998. Following ligand binding to death receptors, the death domain recruits (directly or in the case of TNF-R1 indirectly via TRADD) an adaptor protein, Fas associated death domain (FADD). FADD can then recruit pro-caspase-8 to the ‘Death Inducing Signal Complex’ (DISC), thereby causing its activation. In addition, “cross-talk” between these pathways has been observed. For example, the Bid protein is a pro-apoptotic BH3-only member of the Bcl-2 family that is essential for Fas-induced apoptosis in some cells such as hepatocytes (Yin, X-M., Gene 369: 7-19, 2006).

Activation of either apoptotic cell death pathway can lead to activation of executioner enzymes, such as caspase-8. Caspase-8 activates caspase-3 and other executioner caspases (caspase-6 and caspase-7) that mediate the systematic demolition of the cell. Other pro-apoptotic enzymes which function in apoptosis include, for example, enzymes such as caspase-9; Omi/HtrA2 (a mitochondrial serine protease; Yang, Q.-H., et al. Genes and Development 17: 1487-1496, 2003), and ubiquitin ligases such as atrogin (Gomes, M. D., et al., Proc. Nat'l. Acad. Sci. USA 98: 14440-14445, 2001; Bodine, S. C., et al., Science 294: 1704-1708, 2001; Jagoe, R. T., et al., FASEB J. 16: 1697-1712, 2002).

In certain types of cells there may be “cross-talk” between the death receptor and mitochondrial-mediated pathways. For example, Bid is a pro-apoptotic BH3-only member of the Bcl-2 family that is essential for Fas-induced apoptosis in hepatocytes (Yin, X. M., J. Mol. Med. 78: 203-211, 2000; Yin, X. M., Cell Research 10: 161-167, 2000).

Apoptosis comprises synthesis and/or activation and/or assembly of multiple structures. For example, pro-apoptotic complexes such as apoptosomes can form during apoptosis, and can comprise molecules such as APAF-1 (Soengas, M. S., et al., Science 284: 156-159, 1999), Bcl-2 family members such as Bad, Bax, Bid, Bim, Bak, “Second Mitochondrial Activator of Caspases/Direct Inhibitor of Apoptosis Binding protein with Low pl” (Smac/DIABLO) and puma. Apoptotic cell death can also involve Death-Inducing Signaling Complex (DISC) components. Apoptosis also involves pro-apoptotic signaling pathways (information flow). Molecules involved with these pathways include adaptor molecules such as DAP12 (Takahashi, K., et al, J. Exp. Med. 201: 2005), MyD88 (Liebermann, D. A., et al., Oncogene 17: 3319-3329, 1998); obligate scaffolds such as FADD (Chinnaiyan, A. M., et al., Cell 81: 505-512, 1995), DAXX Yang, X. et al., Cell 89: 1067-1076, 1997); death-inducing kinases such as ASK1 (Ichijo, H., et al., Science 275: 90-94, 1997); cytosol-sequestered transcription factors such as SMAD3 (Kretzschmar, M. et al., Genes and Development 11: 984-995, 1997), FOXO3a (Brunet A. et al., 21: 952-965, 2001); and modulators of G-Protein coupled receptor (G-PCR) activity such as β-arrestins (Attramadal, H., et al., J. Biol. Chem. 267: 17882-17890, 1992); protein phosphatases such as PP2A (Santoro, M. F., et al., J. Biol. Chem. 273: 13119-13128, 1998; Alvarado-Kristensson, M. et al., J. Biol. Chem. 280: 6238-6244, 2005.); and stoichiometric factors such as carboxyl terminal modulator protein (CTMP) (Maira, S. M., et al., Science 294: 374-380, 2001).

In sepsis, apoptotic cell death is a key pathophysiologic event. The dramatic loss of lymphocytes and other immune effector cells in sepsis severely compromises the immune competence of patients with sepsis and results in their inability to eradicate the invading pathogens and renders them more susceptible to secondary hospital acquired infections.

Overexpression of the anti-apoptotic gene bcl-2 can prevent death of immune cells in sepsis and improve survival (Hotchkiss R. S., et al. J. Immunology 162: 4148-4156, 1999; Hotchkiss, R. S., et al., Proc. Nat'l. Acad. Sci. USA 96: 14541-14546, 1999 herein incorporated by reference in their entirety).

RNAi and siRNA

Small interfering RNA (siRNA), also known as short interfering RNA or silencing RNA, are double stranded RNA molecules which can be from about 19 to about 25 nucleotides in length. Among the properties of an siRNA is an ability to mediate reduction or silencing of gene expression in a cell, through a process known as RNA interference (RNAi).

For example, treatment of septic mice with siRNA against genes of the death receptor pathway protein Fas or the executioner protein caspase-8, led to decreased mRNA and protein levels of Fas and caspase-8, respectively, as well as decreased apoptosis in liver and spleen but not thymus (Wesche-Soldato, D., et al., Blood 106: 2295-2301, 2005). In another example, silencing of Fas, but not caspase-8, in lung epithelial cells using siRNA was found to ameliorate pulmonary apoptosis inflammation and neutrophil influx after hemorrhagic schock and sepsis (Perl et al., Am. J. Path. 167: 1545-1559, 2005).

However, protection of organisms or cells from apoptotic cell death through siRNA inhibition of expression of an apoptosis gene other than Fas or caspase-8, such as siRNA inhibition of expression of a protein which contributes to the mitochondrial apoptosis pathway, has not been disclosed or suggested.

### SUMMARY

The present inventors have developed compositions and methods for protecting organisms from cell depletion that can occur in connection with sepsis. The methods can lead to a reduction of apoptotic cell death in a mammal or other animal, such as a human subject in need of treatment of sepsis. In some configurations, the methods can provide down-regulation of one or more pro-apoptotic proteins other than Fas or caspase-8, such as a pro-apoptotic component of the mitochondrial apoptosis pathway, or a pro-apoptotic component of the death receptor pathway. In some configurations, the down-regulation can be of one or more pro-apoptotic proteins of the Bcl-2 family, such as, without limitation, a “BH3-only” protein such as bim, bax, bak or puma. In particular, the inventors have found that down-regulation of pro-apoptotic proteins of the Bcl-2 family can be protective against bacterial-induced cell depletion in vitro, and against sepsis-induced cell depletion in vivo.

Hence, in various aspects, the methods can comprise effecting a down-regulation of pro-apoptotic Bcl-2 protein expression or function at any level of regulation, including transcription, translation, and protein structure or function. In various configurations, the methods comprise administration of at least one siRNA against pro-apoptotic genes to a cell or organism, such as a subject in need of treatment of sepsis. In various configurations, the siRNA can be directed against any pro-apoptotic gene other than Fas or caspase-8. In some aspects, the siRNA can be directed against a gene encoding a protein which contributes to the mitochondrial apoptosis pathway, such as, without limitation, a pro-apoptotic Bcl-2 protein such as Bad, Bik, Bid, Puma, Bim, Bmf, Noxa, Hrk, bax, bak or combinations thereof. In some aspects, a plurality of siRNAs can be administered simultaneously and/or in a combination, such as a combination of siRNAs directed against bax and bak.

In yet other aspects, the methods comprise administration of at least one siRNA against a gene encoding a component of the death receptor pathway, such as a Fas-associated death domain (FADD).

In other aspects, protection from apoptosis can be effected by administration of a targeting moiety conjugated to an anti-apoptotic homology domain of a protein from the Bcl-2 family. Thus, use of compositions as described herein provides novel and potent therapeutic approaches to the treatment of sepsis.

Accordingly, in some aspects, the present teachings provide methods and compositions for treating sepsis. In various configurations, these methods comprise administering to a subject a therapeutically effective amount of a composition comprising an anti-apoptotic siRNA, other than an siRNA against Fas or caspase-8. A composition of the present teachings can comprise an siRNA which can be, in some configurations, an siRNA against a mitochondrial apoptosis pathway component. In some configurations, the siRNA can be directed against a pro-apoptotic Bcl-2. A composition which can be used in these methods can be a pharmaceutical composition, and can further include a delivery vector, vehicle or carrier, such as, without limitation, a liposome, a viral vector, or a nanoparticle. In some configurations, a delivery system can include a cyclodextrin polymer, such as a cyclodextrin-containing polycation. In some configurations, a delivery system can include nanoparticles, such as nanoparticles comprising a cyclodextrin-containing polycation that is decorated with stabilizing agents and/or targeting ligands. In some configurations, the nanoparticles can include surface-modifying agents having terminal adamantane groups. In some aspects, a surface-modifying agent can form inclusion complexes with a cyclodextrin, and a nanoparticle can contain poly(ethylene glycol) (PEG). In some aspects, a delivery vehicle can comprise a cationic lipid, such as 1,2-dioleoyl-3-trimethylammonium propane (DOTAP). In a some configurations, delivery methods of an siRNA can include, but are not limited to, administration of liposomes for liposome-mediated transfection, administration of a viral vector such as a lentivirus vector, an adenovirus vector or an adeno-associated virus (AAV) vector, chemical delivery, or administration of nanoparticles.

In some aspects, a chemical delivery system can use a chemical targeting moeity. A chemical targeting moiety of these aspects can comprise a chemical modification of a 2′-OH of a nucleobase. In some configurations, a chemical targeting moeity can be a polymer, such as a cationic or anionic polymer. In some configurations, a cationic polymer can be a polyethyleneimine (PEI), a poly(L-lysine) (PLL) or a polyamidoamine (PAMAM) such as a PAMAM dendrimer.

In some configurations, a viral delivery vector of a composition of the present teachings can be an intact, attenuated or non-replicative form of a virus such as; without limitation, FIV, HIV, or MMULV.

In other aspects, the inventors provide methods for treating sepsis. In various configurations, these methods comprise administering to a subject a therapeutically effective amount of a composition comprising an siRNA which hybridizes to a gene in an apoptotic pathway. An anti-apoptotic siRNA of these aspects can comprise a nucleotide sequence which hybridizes to any pro-apoptic gene, such as a gene encoding a protein in the mitochondrial pathway and/or the death receptor pathway and/or an execution enzyme, such as but not limited to a pro-apoptotic bcl-2 family member such as Bad, Bik, Bid, Puma, Bim, Bmf, Noxa or Hrk.

In other aspects, the inventors disclose methods of delivering an siRNA to a cell or organism via a liposomal delivery system. In some configurations, a liposomal delivery system comprise an MLV, an SUV, a cationic lipid, an anionic lipid, a synthetically modified lipids, or a combinations thereof. In some configurations, a liposomal delivery system can include one or more functional groups. A functional group can be deposited into the lipid bilayer of a liposome. Without limitation, a function group of these configurations can be, for example, a protein such as an antibody. Furthermore, a liposomal delivery system of these aspects can comprise DOPE, an immunoliposome or PEG.

In additional aspects, the present teachings provide methods of providing protection against sepsis in a mammal, such as a human subject in need of treatment. In various configurations, these methods can comprise preventing assembly of pro-apoptotic complexes by administering to the mammal a composition comprising one or more siRNA against components of the mitochondrial apoptosis pathway and/or the death receptor apoptosis pathway.

In still further aspects, the present teachings provide methods of protecting a cell or organism against sepsis. These methods can comprise inhibiting pro-apoptotic enzyme activity.

In yet further aspects, the present teachings provide methods of protecting against sepsis that involve disrupting information in pro-apoptotic pathways in order to prevent assembly of pro-apoptotic complexes. In some configurations, disruption of information in a pro-apoptotic pathway can be effected by inhibiting phosphorylation that comprises an apoptosis signaling mechanism.

In another aspect, the present teachings encompass methods of protecting against sepsis that involve down-regulating negative regulators of cell survival.

In other aspects, the present teachings includes methods for down-regulating the function or expression of pro-apoptotic genes or proteins in addition to administering siRNA. These methods include, but are not limited to, methods for effecting down-regulating transcription, translation and protein activity. Some of these methods include: administration of an antisense RNA [PMID 2027015]; administration of an shRNA; administration of a PNA; administration of a minor groove targeting agent directed against genomic DNA, such as, for example, a small molecule polyamides [PMID: 16101489]); administration of a major groove of targeting agent directed against genomic DNA, such as, for example, an artificial transcription factor such as a Zn-finger protein [PMID: 11821858]).

Further scope of the applicability of the present invention will become apparent from the detailed description and drawings provided below. However, it should be understood that the following detailed description and examples, while indicating preferred embodiments of the invention, are given by way of illustration only since various changes and modifications within the spirit and scope of the invention will become apparent to those skilled in the art from this detailed description.

### BRIEF DESCRIPTION OF THE DRAWINGS

The present teachings will be better understood from the following detailed description and drawings, which are given by way of illustration only, and are not intended to limit the scope of any claim.

FIG. 1 shows the effect of overexpression of Bcl-xL on sepsis survival in transgenic mice overexpressing Bcl-xL compared to matched wild type mice.

FIG. 2 shows the effect of TAT-Bcl-xL on bacterial-induced human lymphocyte apoptosis. Staining for active caspase 3 shows the percent apoptosis in human lymphocytes co-cultured with E. coli and treated with Tat-Bcl-xL or free unconjugated Bcl-xL.

FIG. 3 shows percent apoptosis in human lymphocytes incubated with E. coli for approximately five hours and then treated with 200 nM, 500 nM or 1 μM TAT-BH4, or 1 μM of the inactive TAT-BH4(D)₂.

FIG. 4 shows the internalization of TAT-BH4 into human lymphocytes. The cells were incubated with TAT-BH4 conjugated with ITEM fluorescein (right panel) and imaged using laser scanning confocal microscopy at 200× magnification. The left panel shows untreated cells.

FIG. 5 shows die results from an in vivo experiment with TAT-BH4. Splenocytes (top panel), thymi and blood (bottom panel) %% ere examined for apoptosis via staining for active caspase 3.

FIG. 6 shows the effect of bim knock out (Bim KO mice) on apoptosis. FIG. 6A is a bar graph of results from wild type (WT) mice and Bim KO mice of active caspase-3 staining in thymus, and FIG. 6B is a bar graph of results of active caspase-3 staining in spleen, showing effect of Bim knock out on sepsis-induced B and T cell lymphocyte apoptosis.

FIG. 7 is a survival curve comparing survival of wild type (WT) and Bim KO mice in sepsis.

FIG. 8 shows mice were treated with 5 nanomolar siRNA to bim or with the control non-coding siRNA. Approximately 24 hrs later, mice underwent sham surgery or cecal ligation and puncture (CLP) to induce sepsis. A third group of mice had vehicle as a second control. Twenty four hrs after sham or sepsis surgery, mice were killed and thymi or spleens were harvested. The cells were dissociated and stained for TUNEL as a measure of apoptotic cell death. Flow cytometry was performed to quantitate apoptosis in CD3 T cells. Note the increase in apoptosis (increased % apoptosis via TUNEL assay—on Y axis) in the CLP mice (septic) compared to the sham mice. Note also that the mice that received siRNA to Bim had a decrease in sepsis-induced apoptosis compared to septic mice receiving vehicle or the non-coding siRNA.

FIG. 9 shows the B cells of the same mice discussed in FIG. 8.

FIG. 10 shows cells from mice were pretreated with siRNA to bim or the inactive non-coding siRNA and sepsis vas induced by cecal ligation and puncture (CLP) 24 hrs later. Survival was followed for 7 days.

FIG. 11 (=FIG. 1 of letter) illustrates prevention of cell loss and decreased apoptosis of spleen cells in vivo by administration of siRNA to bim in sepsis.

FIG. 12 (=FIG. 2 of letter) illustrates prevention of cell loss and decreased apoptosis of thymus cells in vivo by administration of siRNA to bim in sepsis.

FIG. 13 (=FIG. 3 of letter) illustrates amelioration of sepsis-induced decrease in absolute cell numbers of macrophages, dendritic cells and natural killer cells in vivo be administration of siRNA to bim.

FIG. 14 (=FIG. 4 of letter) illustrates presentation of absolute numbers of CD4 T cells in spleen in vivo by treatment With siRNA to PUMA or Bim.

FIG. 15 (=FIG. 5 of letter) illustrates preservation of absolute numbers of neutrophils in spleen in vivo by treatment with siRNA to PUMA or Bim.

FIG. 16 (=FIG. 1 of Bim siRNA paper) illustrates that cationic liposomes yield high transfection efficiency. (A) Autofluorescence of liposome-treated (vehicle without siRNA) human lymphocytes. (B) Liposome-mediated delivery of 5-carboxyfluorescein labeled siRNA to Bim yielded an average eight-fold increase in mean fluorescence intensity (p<0.0001). Data presented as dot plots. Different transfection efficiencies correlate with different fluorescence intensities with the highest peak corresponding to greater uptake of labeled siRNA molecules (C). Data presented as histogram.

FIG. 17 (=FIG. 2 of Bim siRNA paper) illustrates that Bim siRNA abrogates radiation-induced apoptosis in vitro. Quantification of human peripheral blood T-cell apoptosis by TUNEL revealed a return to near baseline levels of apoptosis after treatment with Bim-targeted siRNA (p<0.01).

FIG. 18 (=FIG. 3 of Bim siRNA paper) illustrates qualitative validation of low-volume liposome-mediated delivery of siRNA in vivo. Representative fluorescent images of fresh tissue sections from green fluorescent protein (GFP) transgenic mice (n=5) demonstrated a significant decrease in fluorescence intensity in the spleen and thymus after treatment with GFP-targeted siRNA. There was no decrease in fluorescence intensity after treatment with vehicle or non-targeting siRNA. 200× magnification.

FIG. 19 (═FIG. 4 of Bim siRNA paper) illustrates that Bim siRNA suppresses splenic Bim protein expression in vivo. Representative data showing Bim protein expression in septic mice. Mice treated with Bim-targeted siRNA demonstrated a 33.1±2.9% decrease in Bim protein expression in (A) B cells and a 19.5±2.5% decrease in Bim protein expression in (B) T cells (p<0.01 each). There was no decrease in Bim protein expression after treatment with vehicle or non-targeting siRNA.

FIG. 20 (═FIG. 5 of Bim siRNA paper) illustrates that Bim siRNA protects splenic and thymic lymphocytes from sepsis-induced apoptosis. Isolated splenocytes and thymocytes from sham- or CLP-operated mice were labeled with fluorescent cell-specific markers for B and T cells (b220+ and CD3+ respectively) and for apoptosis (TUNEL). Quantification by TUNEL revealed a return to baseline levels of apoptosis in splenic B cells (A) and T cells (B) in the Bim siRNA treated groups. Quantification by TUNEL also revealed protection against apoptosis in thymic T cells although not complete (C).

FIG. 21 (=FIG. 6 of Bim siRNA paper) illustrates that Bim-targeted siRNA improves survival in a murine model of septic peritonitis. C57BL/6 male mice underwent CLP to induce sepsis. One group received a single daily i.v. injection of Bim-targeted siRNA on peri-injury days −1, 0 and +1 (n=16). The control group received a single daily i.v. injection of non-targeting siRNA on peri-injury days −1, 0 and +1 (n=16). Survival was recorded for 7 days. Mice receiving Bim-targeted siRNA had an overall survival advantage of 90% whereas control animals had on overall survival of only 50% (p<0.03).

FIG. 22 (=FIG. 3 of poster) illustrates reduction in apoptotic cell death in animals receiving siRNA to Bax and Bak.

FIG. 23 (=FIG. 6 of poster) illustrates uptake of fluorescently-labeled siRNA via electroporation.

FIG. 24 (=FIG. 5 of poster) illustrates that a combination of siRNAs against Bax and Bak can protect cells from apoptotic cell death in vitro.

### DETAILED DESCRIPTION

The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Methods of generating and introducing siRNA molecules into cells and organisms can be found in standard texts such as Sohail, M., Gene Silencing by RNA Interference: Technology and Application, CRC Press, 2005; Appasani, K., RNA Interference Technology From Basic Science to Drug Development, Cambridge University Press, 2005; Engelke, D. R., et al., Methods in Enzmology 392, Academic Press, 2005. Methods of formulating drugs can be found, for example, in Hoover, John E., Remington's Pharmaceutical Sciences, Mack Publishing Co., Easton. Pa. (1975); Liberman, H. A. and Lachman, L., Eds., Pharmaceutical Dosage Forms, Marcel Decker, New York, N.Y. (1980); and Goodman & Gilman's The Pharmacological Basis of Therapeutics, 1996, Ninth Edition, McGraw-Hill, New York.

As used herein, the singular forms “a”, “an” and “the” are intended to include the plural forms as well, unless the context indicates otherwise. All publications, patent applications, patents, and other references mentioned herein are incorporated by reference in their entirety.

As used herein, the term “animal” includes, but is not limited to, mammals, including human beings. Furthermore, the compositions and methods disclosed herein are applicable in both human and veterinary medicine. Thus, the present compositions and methods can be applied to mammals, including humans, domestic pets such as cats, dogs, rodents, and birds, farm animals such as cows, sheep, goats, pigs, chickens and horses, and to zoo animals.

As used herein, the term “BH4” broadly refers to an anti-apototic homology domain of a Bcl-2 family member protein, one of four homology domains as described, for example, in Science, 281, 1322-26 (1998).

As used herein, the term “Tat-BH4” refers to a peptide having the following amino acid sequence (d)-Ac-RKKRR-Om-RRR-bAla-(l)-SNRELVVDFLSYKLSQKGYS-COOH (SEQ ID NO: 1), wherein bAla represents β-alanine, Orn is ornithine, and the N-terminus is acetylated, and also encompasses the same peptide using (l)-amino acids or mixtures of (l)- and (d)-amino acids, and also the same peptide in which ornithine is replaced by glutamine, i.e., (d)-Ac-RKKRRQRRR-bAla-(l)-SNRELVVDFLSYKLSQKGYS-COOH (SEQ ID NO: 2), and also the same peptide in which the N-terminus is not acetylated, and also the same peptide comprising retro-inverso sequences or more than one of the variations as listed herein.

Amino acids are indicated herein using the single letter notation conventional in the art. When used in amino acid sequences, the letter “x” designates any amino acid. When used in an amino acid sequence, a “/” between two adjacent letters indicates that either of the amino acids listed can be used. When used in nucleotide sequences, the letter “n” designates A, T, C or G in the case of a deoxyribonucleotide, or AUCG in the case of a ribonucleotide.

### Therapy of Sepsis by Preventing Cell Death Via Manipulation of the Bcl-2 Family

Shown herein are mice that have knockout of pro-apoptotic genes (genes that cause apoptotic cell death), which are almost completely resistant to apoptotic cell death and have a markedly improved survival in sepsis (for example, see Example 14). As supported by these results, applicants have devised methods for controlling sepsis in human subjects, namely through the control the expression of pro-apoptotic genes, such as pro-apoptotic bcl-2 family members. These methods comprise administration of siRNA directed against pro-apoptotic genes such as pro-apoptotic bcl-2 family members. Included within the present teachings are methods of delivery, of these siRNA molecules to cells in vivo.

In another aspect, targeted cell death of infected immune cells can be accomplished by delivers of siRNA to pro-apoptotic family members using molecules conjugated to the siRNA, or methods utilizing liposomes and other delivery methods (detailed below) that target delivery to particular types of cells. Using the concepts and methods set forth herein, cell death can be prevented in specific classes of immune cells during various infections. For many infections, various classes of immune cells, such as various lymphocyte subsets (e.g., CD4 cells, CD8 cells, B cells), dendritic cells, and/or monocytes can be targeted with therapeutic compounds to specific cell types/tissues using molecules that selectively or specifically recognize cell surface markers whose expression is either restricted or increased on a subset of cells. Molecules that confer cell type selectivity or specificity include but are not limited to: naturally arising antibodies or antibody fragments, in vitro expressed and/or affinity matured antibody-mimics (for example scFv), recombinant protein ligands or domains derived from those molecules that confer selectivity or specificity, small molecule ligands or in the case of pattern recognition receptors such as the TLRs, natural, semi-synthetic or synthetic ligands to those receptors, so that one can modify the innate and adaptive immune system for therapeutic applications.

In various aspects of the present teachings, methods of treating or preventing sepsis include downregulating one or more components of apoptosis pathways. These components can include, for example: components of pro-apoptotic complexes, such as, without limitation, APAF-1; pro-apoptotic Bcl-2 family members such as Bim, Bad, Bax, Bid, Smac/DIABLO, and DISC; pro-apoptotic enzymes, such as, without limitation, Caspases including Casp-3, Casp-7, Casp-8, and Casp-9, Omi/HtrA2, and ubiquitin ligases such as atrogin; components of pro-apoptotic signaling pathways, such as, without limitation, DAP12, and MyD88; obligate scaffolds such as, without limitation, FADD and DAXX; death-inducing kinases such as, without limitation, ASK1; cytosol-sequestered transcription factors such as, without limitation, SMAD3 and FOXO3a; modulators of G-PCR activity such as, without limitation, β-arrestins, and negative regulators of survival pathways, such as, without limitation, enzyme such as protein phosphatase 2A (PP2A); and siRNA against stoichiometric factors such as carboxyl-terminal modulator protein (CTMP).

As further explanation, we describe apoptotic pathways that can be down-regulated via modulation of transcriptional, translational, and/or protein levels. These methods include, but are not limited to, inhibiting gene expression by administration of siRNA.

### Other Delivery Methods

Nucleic acid delivery to cells in vivo and in vitro (including, but not limited to nucleic acids such as DNA/RNA (including RNAi)) can be accomplished through a number of biological, chemical and/or physical targeting techniques. Physical targeting methods are used to deliver the material directly to the site of action. This can include direct injection into a tissue via a number of methods including but not limited to, intramuscular, intracerebral or body cavity (peritoneum); implantation of an access port (central line); inhalation; and topical administration. Physical targeting can be coupled with other targeting methods, for example delivering the molecules discussed below to a physical location in an organism, including a human.

Chemical targeting includes methods to mask the activity of the compound or direct the uptake of the compound using synthetic molecules. Pro-drug strategies for nucleic acid deliver) include nucleotides chemically modified at one or more of the 2′-OH or the nucleobase. These modifications are designed to be enzyme-labile, and thus, the materials are activated only in cells that express the enzyme of interest.

Biological targeting includes incorporation of native ligands (or engineered mimetics) to direct the uptake of a therapeutic agent into target cells that express the cognate receptor. Receptors recognize a wide range of endogenous and exogenous molecules including, but not limited to, peptides, mono-, oligo-, or poly-saccharides, nucleotides and nucleosides, proteins, pathogen associated molecular patterns, hormones, and other naturally occurring, semi-synthetic or synthetic molecules. Antibodies, antibody fragments or engineered antibody-mimetics are also effective targeting moieties.

Liposomes, nanoparticles (fluorocarbon emulsions, SNALPs), viral vectors and chemical encapsulation techniques (engineered cyclodextrin derivatives) can be used in the invention. Viral vectors provide the additional control of transient or persistent expression of the siRNA. Viral vectors also provide the additional control of using tissue-specific promoters to further improve cell-type specificity. Naked siRNAs or backbone modified RNAs are also used effectively to deliver nucleic acids, including siRNA.

Adenoviral vectors also function effectively to deliver nucleic acids to cells. Adenoviral vector entry into the cell, or transduction, involves a number of interactions between proteins on the capsid coat of the vector and target cell surface. Transport to the nucleus proceeds where DNA replication and transcription occur from an epichromosomal location, resulting in expression of the gene of interest (GOI). Adenoviral vectors will function to deliver nucleic acids of the invention.

Retroviruses are an efficient means to deliver single DNA expression constructs to a wide range of mammalian cell types. They are efficient delivery vehicles for nucleic acids of the present invention. As examples, three systems are discussed, but retroviral vectors useful in the present invention include all retroviruses, not just those discussed as examples. Vectors based on Moloney Murine Leukemia Virus (MMULV) allow for delivery of genes to most dividing mammalian cell types. Vectors based on Feline Immunodeficiency Virus allow for delivery of genes to most mammalian cell types (including dividing and nondividing cells). The third system is based on HIV-1 and allows for delivery of genes to most dividing and non-dividing mammalian cell types with very high efficiency.

Commonly used nonviral vectors for delivery of nucleic acid-based therapeutics can be classified into 2 major types based on the nature of the synthetic material: i) polymeric delivery systems (nucleic acid-polymer complexes) and ii) Liposomal deliver; systems (DNA entrapped in and/or complexed to liposomes). “Polymer,” as used herein, refers to a polymer that is neither a polypeptide nor a polynucleotide.

In various configurations, cationic polymers can be used in gene delivery because their can easily complex with the anionic nucleic acid molecules. PEI (“Polyethylenimine”) is a branched polymer with high cationic potential that is capable of effective gene transfer in mammalian cells. PLL (“Poly(L-lysine)”) is a biodegradable cationic polymer that is used to deliver DNA-based therapeutics such as oligonucleotides. Chitosan is a natural biodegradable polymer that is an alternative to PEI and exhibits low toxicity. Polyamidoamine (PAMAM) dendrimers represent a recently developed class of polycationic synthetic polymers that can be used for gene transfer.

In some aspects of the present teachings, liposomes can be used for the efficient delivery of a nucleic acid such as an siRNA to cells. Liposomes are vesicles that comprise an aqueous compartment enclosed in a phospholipid bilayer. Liposomes, as used herein, can include multilamellar vesicles (MLV) comprising multiple bilayers of lipids formed around a primary core in a concentric fashion. Liposomes can also include small unilamellar vesicles (SUV). In some configurations, an SUV can be in a size range from about 100 nm up to about 500 nm diameter.

In some configurations, a variety of cationic, anionic, synthetically modified lipids, and combinations thereof can be used to deliver a nucleic acid of the present teachings.

In some aspects, a nucleic acid of the present teachings can be delivered using pH-sensitive liposomes. These liposomes can be generated by the inclusion of dioleoylphosphatidylethanolamine (DOPE) into liposomes composed of acidic lipids such as cholesterylhemisuccinate or oleic acid, as described, for example, by Hong, M.-S., et al, J. Pharmacy Pharmacol. 54: 51-58, 2003, or Bergstrand, N., et al., Biophys. Chem. 104: 361-379, 2003.

In some configurations, immunoliposomes, which incorporate antibodies attached to lipid bilayers, can also be used as deliver vehicles for nucleic acids of the present teachings. Immunoliposomes can be prepared and administered using methods known to skilled artisans, such as methods described by Wang, C. Y., et al., Proc. Nat'l Acad. Sci. USA 84: 7851-7855, 1987; Maruyama, K., et al., Proc. Nat'l Acad. Sci. USA 87: 5744-5748, 1990; Huwyler, J., et al., Proc. Nat'l Acad. Sci. USA 93: 14164-14169, 1996; Pirollo, K. F., et al., Hum Gene Ther. 17: 117-124, 2006; Pirollo, K. F., et al., Cancer Research 67: 2938-2943, 2007.

In some configurations, stealth liposomes, can also be used as deliver) vehicles for nucleic acids of the present teachings. Stealth liposomes can be prepared and used by methods known to skilled artisans, such as, for example, the methods set forth in Foged, C., et al., Int. J. Pharm. 331: 160-166, 2007.

In some configurations, permeant peptides, such as Tat, as described in members of the patents family to which this application claims priority, can also be used for delivery.

Conjugated siRNA and miRNA's

The present invention also encompasses the use of targeted gene silencing RNA sequences in the compounds, such as short interfering RNA (siRNA). siRNA's are short (about 19 to about 25 nucleotides long) double-stranded RNA sequences known to be useful for silencing specific genes. In some configurations, the present methods include compositions comprising cell membrane-permeant compounds and anti-apoptotic siRNAs, such as siRNA directed against expression of a pro-apoptotic Bcl-2 protein such as Bim.

The present inventors have developed methods and compounds that use siRNA in a treatment approach for sepsis. In some aspects, an siRNA can be introduced into cells using a transfection agent. In other aspects, an siRNA can be administered by i.v. injection as a bare nucleic acid or complexed with lipids. In yet other aspects, an siRNA can be introduced in vivo by administering a mixture comprising the siRNA and a protamine-Fab (antibody) fusion protein (Song, E., et al., Nat Biotechnol 23: 709-717, 2005) which is herein incorporated by reference in its entirety). In some configurations, a composition of the present teachings comprises a cell membrane-permeant peptide such as Tat conjugated to an anti-apoptotic siRNA, such as an anti-Bim siRNA, the sequence of which is determined by reference to the known human sequences for BCL2L11 (GenBank Accession No. NM_{—}006538), set forth herein as SEQ ID NO: 8, as well as transcriptional variants thereof. siRNA's against specified sequences are commercially available or can be synthesized using known oligonucleotide synthetic techniques. In an exemplary embodiment, an siRNA can be coupled to Tat or other cell membrane-permeant peptide via a covalent bond. For example, a Tat-(anti-Bim-siRNA) heterodimer may be formed through the formation of a thioether bond. Bim-directed siRNA will be delivered intracellularly for silencing of Bim, effectively targeting cells expressing Bim, such as lymphocytes.

In some configurations, the present teachings include other covalent or non-covalent association of an siRNA with membrane permeant peptides such as Tat. For example, compounds can be made to provide stoichiometric or super-stoichiometric delivery of siRNA. TAT can be conjugated with a polycationic molecule such as protamine, to produce a non-covalent compound having a stoichiometry of about six (6) siRNA per conjugate. TAT can be directly conjugated to siRNA through a linear structure to produce a covalent compound such as a TAT-siRNA having a stoichiometry of one (1) siRNA per conjugate. TAT can also be conjugated through a branching structure to produce a covalent compound such as a TAT-Lysine(aNH2, eNH2)-siRNA(2) having a stoichiometry of two (2) siRNA per conjugate. Compounds using higher order branched structures can be made to deliver 2ⁿ siRNA/conjugate, where n=number of branch points.

In some configurations, methods and compounds are disclosed for detaching an siRNA from a membrane permeant peptide once the compound is inside the cell. Such compounds, for example, include a functional linker such as a protease-reactive sequence for linking the siRNA to TAT or other membrane permeant peptide. Suitable peptide sequences can be, for example, those recognized by interleukin-1β converting enzyme (ICE) homologues, such as the DEVD amino acid sequence that is recognized by active caspases. For example, such a compound can be a TAT-DEVD-siRNA compound. The DEVD sequence can be cleaved by caspases active within the cell, leaving the siRNA within the cell, while TAT leaves the cell. The compounds therefore also therefore provide a method to separate siRNA cargo from the membrane permeant peptide component such as TAT.

### Pharmaceutically Acceptable Salts of Peptide Complexes

In various aspects, a peptide complex of the present invention can be used in a free acid/base or a peptide salt. A peptide salt can comprise, without limitation, an organic anion, an organic cation, a halide, or an alkaline metals.

The term “pharmaceutically acceptable salts” embraces salts commonly used to form alkali metal salts and addition salts of free acids or free bases. A pharmaceutically acceptable base addition salts of the present peptide complexes include metallic salts and organic salts.

Examples of metallic salts can include, but are not limited to, alkali metal (group Ia) salts, and alkaline earth metal (group IIa) salts. Such salts can be prepared, for example, from aluminum, calcium, lithium, magnesium, potassium, sodium, or zinc.

Examples of organic salts can comprise, but are not limited to, tertiary amines and quaternary ammonium salts, including in part, tromethamine, diethylamine. N,N′-dibenzyl-ethylenediamine, chloroprocaine, choline, diethanolamine, ethylenediamine, meglumine (N-methyl-glucamine), and procaine. Such salts can also be derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate.

The basic nitrogen-containing groups can be quaternized with agents such as lower alkyl halides, such as methyl, ethyl, propyl, and butyl chloride, bromides, and iodides; dialkyl sulfates such as dimethyl, diethyl, dibuytl, and diamyl sulfates, long chain halides such as decyl, lauryl, myristyl, and stearyl chlorides, bromides, and iodides; aralkyl halides such as benzyl and phenethyl bromides, and others.

All of these salts can be prepared by conventional means from the corresponding peptide complex disclosed herein by reacting the appropriate acid or base therewith. Water- or oil-soluble or dispersible products are thereby obtained as desired.

### Formulations/Pharmaceutical Compositions

Compounds used according to the methods of the present teachings can be formulated as pharmaceutical compositions. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known, art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and welling agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary, temperatures but liquid at the rectal temperature, and which will therefore melt in the rectum and release the drug.

Solid dosage forms for oral administration may include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage form will vary depending upon the patient and the particular mode of administration.

### Doses/Quantities of Peptide Complexes

The quantity of a cell membrane-permeant peptide compound comprising an anti-apoptotic protein domain for treating sepsis should be an effective amount for the intended purpose. Such amounts can be determined empirically, and are also well known in the art. Guidance for determining drug dosages for treating various conditions are well known in the art. Note in this regard, for example, Goodman & Gilman's The Pharmacological Basis of Therapeutics, 1996, Ninth Edition, McGraw-Hill, New York. For example, amounts of Tat-BH4 administered via the present complexes can be in the range of from about 5 mg/kg-body-weight/day to about 2000 mg/kg/day, preferably from about 50 mg/kg/day to about 1500 mg/kg/day, and in one embodiment from about 100 mg/kg/day to about 1000 mg/kg/day. This amount can be adjusted for body weight and the particular disease state, and other factors as known in the medical art.

### Routes of Administration

The complexes according to the present methods can be administered by a variety of methods, including, for example, orally, enterally, mucosally, percutaneously, or parenterally. Parenteral administration is preferred, especially by intravenous, intramuscular, subcutaneous, intracutaneous, intraarticular, intrathecal, and intraperitoneal infusion or injection, including continuous infusions or intermittent infusions with pumps available to those skilled in the art. Alternatively, the complexes can be administered by means of micro-encapsulated preparations, for example those based on liposomes as described in European Patent Application 0 213 523.

### Treatment Regimens

The regimen for treating a patient with the compounds and/or compositions of the present invention is selected in accordance with a variety of factors, including the age, weight, sex, diet, and medical condition of the patient, the severity of the condition, the route of administration, pharmacological considerations such as the activity, efficacy, pharmacokinetic, and toxicology profiles of the particular pharmacologically active compounds employed.

Administration of the drug complexes disclosed herein should generally be continued over a period of several day's, weeks, months, or years. Patients undergoing treatment with the drug complexes disclosed herein can be routinely monitored to determine the effectiveness of therapy for the particular disease or condition in question.

Continuous analysis of the data obtained by these methods permits modification of the treatment regimen during therapy so that optimal amounts of the pharmacologically active substance in the peptide complex are administered, and so that the duration of treatment can be determined as well. Thus, the treatment regimen/dosing schedule can be rationally modified over the course of therapy so that the lowest amounts of drug compound is administered, and so that administration of such compounds is continued only so long as is necessary to successfully treat the disease or condition.

### Monitoring Devices/Procedures

Detection methods useful in practicing the present invention include, but are not limited to magnetic resonance, superconducting quantum interference device (squid), optical imaging (e.g. fluorescence tomography, NIRF imaging systems, in vivo bioluminescence, and endoscopic fluorescence), positron emission tomography, and in particular, planar scintigraphy or single photon emission computed tomography (SPECT). Alternative methods of detection include gamma counting, scintillation counting, scanning radiograms, densitometry and fluorography. These detection methods can be employed during or after an effective time interval for diagnosis or imaging subsequent to administering a peptide complex of the present invention. Such effective time intervals are well know in the art, or can be determined be routine experimentation employing methods such as those disclosed herein.

Although the examples hereinafter provided contain many specificities, these should not be construed as limiting the scope of the invention, but as merely providing illustrations of some of the aspects of the present invention.

### EXAMPLE 1

This example sets forth cecal ligation and puncture (CLP) as a model system for sepsis.

Mice that selectively overexpress Bcl-xL in T lymphocytes using the lck-proximal promoter were backcrossed to C57BL6/J (Jackson Laboratory) mice for >10 generations. Tail snips were used to verify presence of the transgene via PCR analysis.

C57BL6/J male mice were housed for at least one week before manipulations. Mice were anesthetized with halothane and an abdominal incision was performed. The cecum was identified, ligated, and punctured with a #30 gauge needle. The abdomen was closed in two layers and 1 cc of 0.9% saline was administered subcutaneously.

The cecal ligation and puncture (CLP) model was used to induce intra-abdominal peritonitis. It has been shown that positive blood cultures for poly microbial organisms (aerobic and anaerobic) result from this model, but not from sham-operated mice. (Baker et al., 1983, Surgery, 94:331; Hotchkiss et al. 2000, Nat Immunol. 1:496).

For survival studies mice received 25 mg/kg of imipenem 3 hours postoperatively and twice per day for two days. Survival was recorded for 7 days. Figure

### EXAMPLE 2

This example illustrates quantification of apoptosis

In these experiments, thymocytes and splenocytes were obtained from CLP and sham-treated mice ˜20 hours postoperatively. The APO-BRDU™ kit (Phoenix Flow Systems, San Diego, Calif.) was employed for flow cytometric quantitation of TUNEL. Antibodies to active caspase 3 (Cell Signaling—Catalog #9664) were used in the flow cytometry and/or TUNEL assay.

Lymphocyte B and CD3 T cells were identified using fluorescently labeled monoclonal antibodies directed against their respective CD surface markers (Pharmingen). Flow cytometric analysis (25,000-50,000 events/sample) was performed on FACscan (Becton Dickinson, San Jose, Calif.).

### EXAMPLE 3

This example illustrates E. coli bacterial-induced lymphocyte apoptosis.

Lymphocytes were harvested from peripheral blood obtained from 6 health) volunteers using a ficol gradient separation technique. Approximately 1×10⁶ lymphocytes were plated in individual transwell containers. E. coli bacteria (strain ATCC 25922), that had been grown overnight in trypticase soy broth were added to a separate compartment of the transwell chamber separated from direct contact with the lymphocytes by a 0.02 micron filter (25 μl of bacteria at 3×10⁹ CFUs added to 1 ml volume.

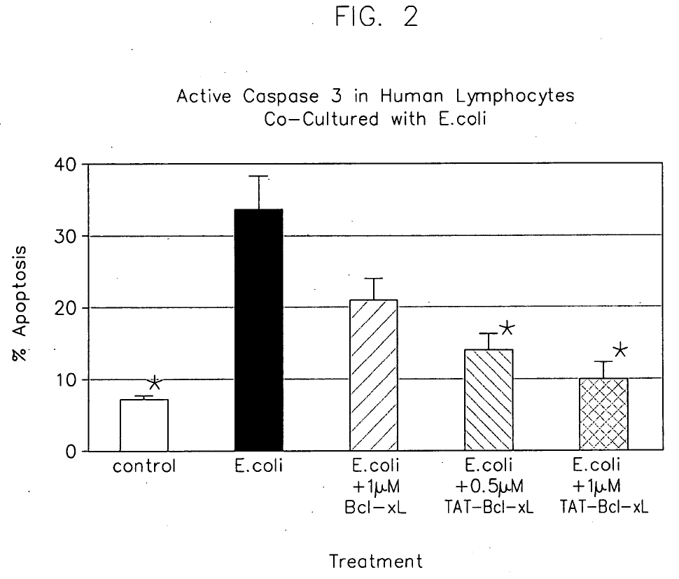

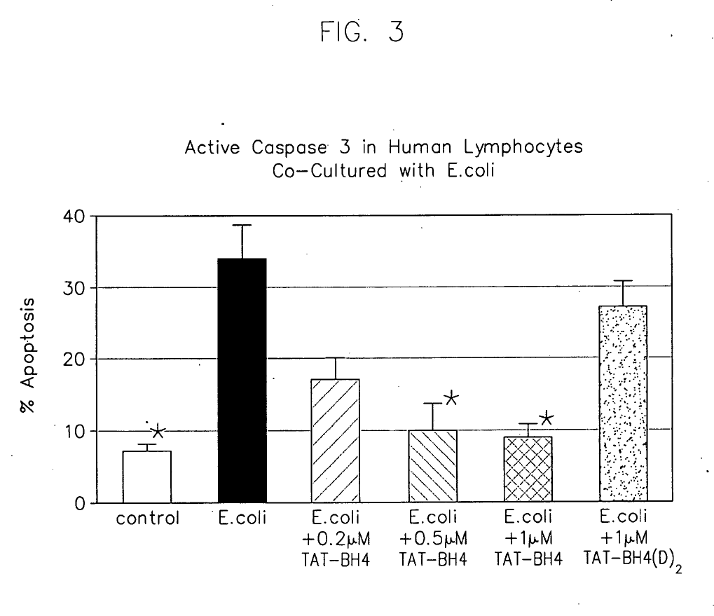

Bcl-xL, TAT-Bcl-xL, TAT-BH4, or an inactive TAT-BH4(D)₂ (d)-Ac-RKKRR-Orn-RRR-bAla-(1)-SNRELVVDFLSYKLSQKGYS-COOH (SEQ ID NO: 1) were placed in experimental wells within 20 minutes after addition of bacteria. The inactive TAT-BH4(D)₂ was identical to TAT-BH4 except that two tyrosines essential for the anti-apoptotic activity of BH4 were replaced by aspartate to render it inactive, and had the sequence (d)-Ac-RKKRR-Om-RRR-bAla-(l)-SNRELVVDFLSDKLSQKGDS-COOH (SEQ ID NO: 3). The lymphocytes were then incubated for 5 hours. Treatment with Tat-Bcl-xL decreased CD3 T cell apoptosis as determined by staining for active caspase 3, but a similar decrease was not seen from treatment with free unconjugated Bcl-xL (FIG. 2). p<0.05. Human lymphocytes (1×10⁶) were treated with live E. coli for ˜5 hours to induce apoptosis. Treatment with 500 nM and 1 μM TAT-BH4 caused a significant decrease in bacterial-induced apoptosis, while the inactive TAT-BH4(D)₂ did not prevent apoptosis (FIG. 3).

### EXAMPLE 4

This example illustrates expression and purification of recombinant TAT-Bcl-xL

In these experiments, the Bcl-xL coding sequence was amplified from C57BL6/J mouse whole-brain cDNA using a polymerase chain reaction procedure. Purified polymerase chain reaction fragments were cloned in the XboI/EcoRI sites of the pTAT-HA vector. All expression cassettes included a sequence encoding six consecutive histidine residues for purification. TAT-Bcl-xL was expressed in E. coli strain BL21 (DE3)pLysS (Novagen, Madison, Wis.) and lysed by sonication. E. coli lysates were denatured in 8M urea prior to affinity chromatography. Bacterial debris was pelleted and the supernatant %% as subjected to metal-affinity chromatography using a Ni-NTA matrix. TAT-Bcl-xL identity was confirmed by Western blotting. Urea and salt were removed by gel filtration using a PD-10 Sephadex G-25M column (Amersham Biosciences, Uppsala, Sweden).

### EXAMPLE 5

This example illustrates peptide synthesis.

In these experiments, amino acid sequences of TAT basic domain and the BH4 peptide employed in the present study are similar to those employed by others in the field with two exceptions.

First, (d)-amino acids were used for synthesis of TAT basic domain for the slower metabolism of these amino acids, leading to a prolonged half-life of the compound.

Second, previous sequence-activity analysis had shown that substitution of ornithine for glutamine enhanced cell permeation of the TAT peptides by ˜10-fold. (see Gammon et al, Bioconjug Chem 14:368).

The amino acid sequence of TAT-BH4 was the following:

(d)-Ac-RKKRR-Om-RRR-bAla-(i)-SNRELVVDFLSYKLSQKGYS-COOH (SEQ ID NO: 1) wherein bAla represents β-alanine, Orn is ornithine and the N-terminus is acetylated.

The peptide used as a control for TAT-BH4 was identical to TAT-BH4 with the exception of two amino acid substitutions: aspartic acid replaced two tyrosines in the BH4 sequence, i.e., (d)-Ac-RKKRR-Orn-RRR-bAla-(l)-SNRELVVDFLSDKLSQKGDS-COOH (SEQ ID NO: 3). These substitutions rendered the BH4 inactive by simulating the native phosphoprotein domain (see Sugioka et al. Oncogene 22:8432).

The amino acid sequence of the inactive TAT-BH4(D)₂ was the following: (d)-Ac-RKKRR-Om-RRR-bAla-(l)-SNRELVVDFLSDKLSQKGDS-COOH (SEQ ID NO: 4).

Peptides were generated by solid phase peptide synthesis using standard Fmoc chemistry by Tufts University Peptide Synthesis Core and purified by HPLC. Identity was confirmed by amino acid analysis and mass spectrometry. Purity was >95%.

### EXAMPLE 6

This example illustrates in vivo administration of TAT-BH4 via infusion pumps

In these experiments, to evaluate the anti-apoptotic efficacy of TAT-BH4 in an in vivo model of sepsis, min-osmotic pumps (Alzet Model 2001D, Durect Corporation. Cupertino, Calif.) were loaded with 1 mg of TAT-BH4 or that TAT-BH4(D)₂ inactive analog dissolved in 200 μl sterile saline and implanted in the subcutaneous tissues on the dorsum of the mice. The pumps were implanted approximately 3 hours prior to CLP as it requires ˜3 hours for pumps to activate and deliver steady state levels of compound. In addition to the TAT-BH4 peptides that were administered by the Alzet mini-osmotic pumps, and additional dose of 0.5 mg of TAT-BH4 or inactive TAT-BH4(D)21 was administered via i.p. injection 2-3 hours prior to sacrifice of the animals which was approximately 18 hours post procedure.

### EXAMPLE 7

This example illustrates laser scanning confocal microscopy of TAT-BH4 treated human lymphocytes

In these experiments, to further functionalize TAT-BH4, a fluorescent label was conjugated to the peptide. To prepare the fluorescently labeled TAT-BH4, (d) ac-C(FM)RKKRR-Orn-RRR-β-A-(l)-SNRELVVDFLSYKLSQKGYS-COOH (SEQ ID NO: 5), an N-terminus cysteine was included in the initial solid state peptide synthesis of the peptide, and FM represents fluorescein maleimide.

Following HPLC purification, the peptide was thiol-conjugated to fluorescein maleimide (FM, 1.2 equiv; Molecular Probes, Eugene, Oreg.) at ambient temperature in 50% DMF/water for 2 hours. Quantitative yields were analyzed by C₁₈ reverse-phase HPLC(RP-HPLC).

Freshly isolated human lymphocytes were incubated with fluorescently labeled Tat-BH4 peptide to confirm intracellular localization of the functional permeant peptide. For labeling, cells were suspended for 30 minutes in modified Earl's balanced salt solution containing 1 μM of the fluorescently labeled TAT-BH4. Control cells were treated identically except no labeled TAT-BH4 was added. Following fixation for 10 minutes in 4% paraformaldehyde, cells were analyzed for peptide internalization via detection of fluorescence by confocal microscopy using an inverted Zeiss Axi overt 200 laser scanning confocal microscope couple to a Zeiss LSM 5 PASCAL fitted with a 488 nm excitation Argon laser and a 520 nm bandpass emission filter. All images were obtained using a water immersion lens (40×) and identical instrument settings.

### EXAMPLE 8

This example sets forth statistical analysis methods

Data are reported as the mean±SEM. Data were analyzed using the statistical software program Prism (GraphPad Software, San Diego, Calif.). Data involving two groups were analyzed by a student's test, while data involving more than two groups were analyzed using one-way analysis of variance (ANOVA) with Tukey's multiple comparison test. Significance was accepted by p<0.05.

### EXAMPLE 9

This example illustrates that overexpression of Bcl-xL prevents lymphocyte apoptosis induced by sepsis.

In these experiments, mice were given cecal ligation and puncture (CLP) or sham surgery. Thymocytes and splenocytes were harvested ˜20-22 hours after surgery.

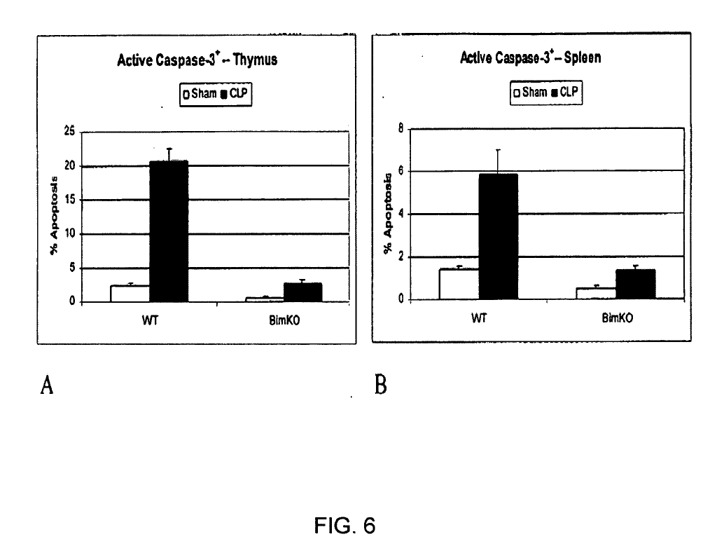

Flow cytometry and staining for active caspase 3 showed that apoptosis was markedly increased in thymocytes (FIG. 6A) and splenocytes (FIG. 6B) in wild type mice that were septic (WT CLP) compared to Bcl-xL mice that were septic (Bcl-xL CLP). p<0.05.

Flow cytometry and TUNEL staining for DNA strand breaks showed that overexpression of Bcl-xL prevented sepsis-induced increase in TUNEL positive cells in both thymus and spleen (p<0.05).

### EXAMPLE 10

This example illustrates that overexpression of Bcl-xL improves sepsis survival

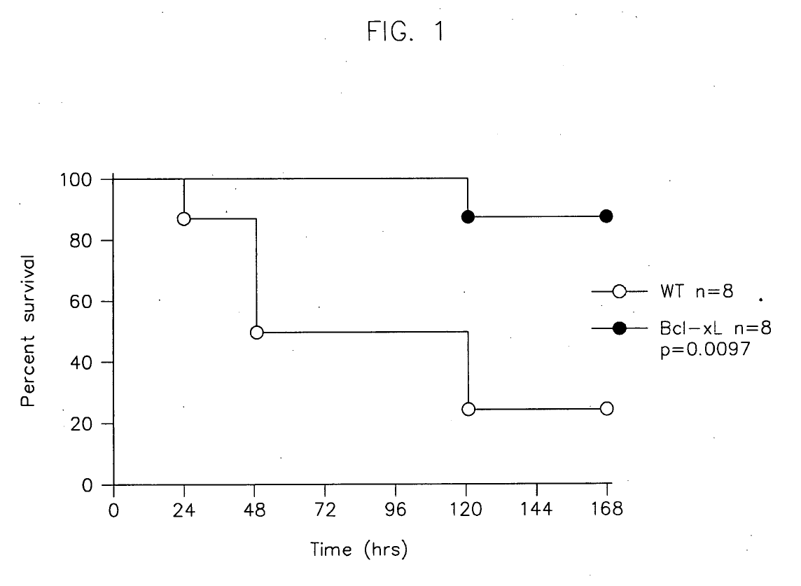

In these experiments, sepsis was induced by CLP in transgenic mice overexpressing Bcl-xL using an lck promoter. Survival was followed for 7 days. The transgenic mice showed improved surival compared to matched wild type C57BL6 mice. (FIG. 1) p=0.097.

### EXAMPLE 11

This example illustrates that human lymphocytes internalize TAT-BH4

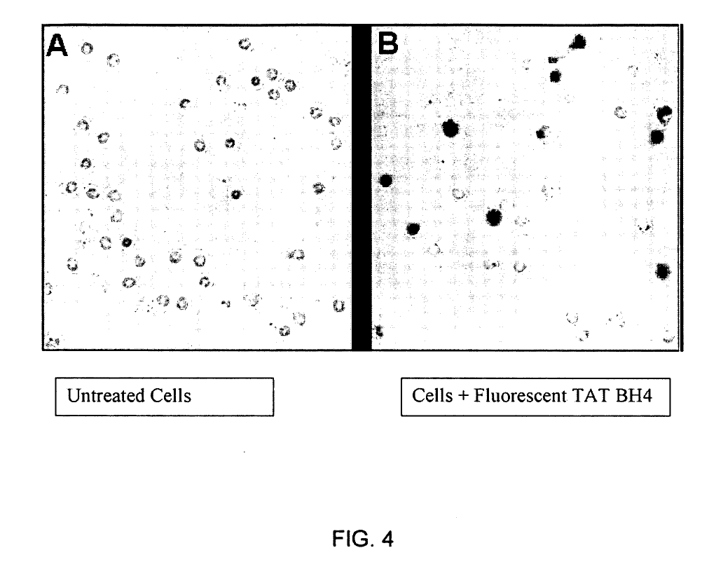

In these experiments, human lymphocytes were incubated in media containing 1 μM fluorescein conjugated TAT-BH4. Laser scanning confocal microscopy demonstrated presence of the fluorescently labeled TAT-BH4 throughout the cell, establishing that the peptide was located intracellular (FIG. 4). Human lymphocytes that were not incubated with the labeled TAT-BH4 conjugated showed minimal autofluorescence and only a faint outline of cells is visible (FIG. 4). 200× magnification. Negative image of fluorescence; originally green-fluorescing cells appears dark against the background.

### EXAMPLE 12

This example illustrates that TAT-BH4 decreases sepsis-induced lymphocyte apoptosis in vivo

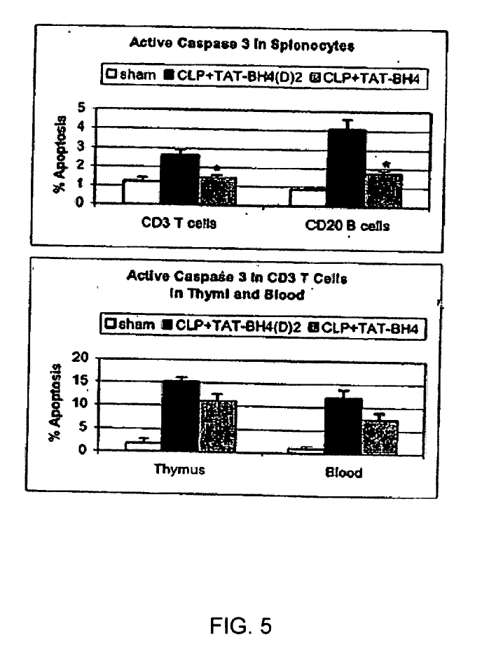

In these experiments, mini-osmotic infusion pumps containing 1 mg of TAT-BH4 or inactive TAT-BH4(D)₂ were implanted in subcutaneous tissues on the dorsum of the mice three hours prior to CLP. The pumps were not activated until approximately three hours after implantation. Mice received an additional 9.5 mg dose of TAT-BH4 or inactive TAT-BH4(D)₂ via i.p. injection two to three hours prior to sacrifice. Spleens, thymi and blood were harvested and examined for apoptosis via staining for active caspase 3. TAT-BH4 ameliorated the increase in sepsis-induced CD3/T cell and B cell apoptosis in the spleen (FIG. 5).

### EXAMPLE 13

This example illustrates that knockout of the pro-apoptotic bim prevents sepsis-induced lymphocyte apoptosis and improves survival

The degree of lymphocyte apoptosis in animal models of sepsis is strongly correlated to survival. Bim, a pro-apoptotic molecule, is essential for lymphocyte deletion during normal homeostasis. Bim induces apoptosis by binding the anti-apoptotic molecules Bcl-2 and/or Bcl-XL on the mitochondrial membrane thereby inhibiting their anti-apoptotic function. The purpose of this study was to compare the degree of lymphocyte apoptosis and survival in Bim −/− versus wild type mice in a clinically relevant model of sepsis. Bim −/− mice were tested to determine whether they show a decrease in sepsis-induced lymphocyte apoptosis and improved survival.

In these experiments, Bim −/− mice and their respective controls (male C57Bl/6 weighing 22-28 gm) were subjected to either cecal ligation and puncture (CLP) or sham surgery (n=63). One cohort (n=32) was sacrificed at 20-22 hrs post surgery and thymi and spleens were harvested for FACS analysis using activated caspase 3 as a marker for apoptosis. A second cohort (n=31) was followed for survival over 7 days.

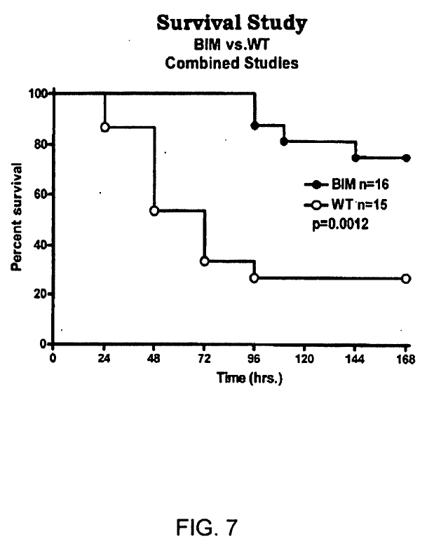

FIG. 6 is a bar graph comparing sepsis-induced B and T cell lymphocyte apoptosis in wild type (WT) and bim knock out (Bim KO) mice. The degree of lymphocyte apoptosis in septic Bim −/− mice approximated that of the sham operated mice indicating near total protection against lymphocyte apoptosis in Bim −/− mice. FACS analysis of thymic lymphocytes demonstrated 20.1+/−2.5% lymphocyte apoptosis in wt CLP mice vs. 2.6+/−0.7% lymphocyte apoptosis in Bim −/− CLP mice (p<0.000003). Likewise. FACS analysis of splenic lymphocytes demonstrated 6.8+/−1.3% lymphocyte apoptosis in wt CLP mice vs. only 1.4+/0.2% apoptosis in Bim −/− CLP mice (p<0.0008). This striking difference in lymphocyte apoptosis correlated with a marked survival advantage in the Bim −/− mice. FIG. 7 is a survival curve comparing survival of wild type (WT) and bim knock out (Bim KO) mice. At 7 days there was 75% overall survival in Bim −/− CLP mice vs. 20% overall survival in wt CLP mice (p=0.0012).

Bim −/− mice have near total protection against sepsis-induced lymphocyte apoptosis and a marked survival benefit (see FIG. 7 for survival benefit).

### EXAMPLE 14

This example illustrates that mice treated with siRNA to bim have a dramatic reduction in sepsis-induced apoptosis of B cells and T cells in the spleen. There was also a trend toward decreased apoptosis in thymus. Treatment significantly increased survival. These data show the efficacy of this approach in preventing cell death in sepsis in human patients.

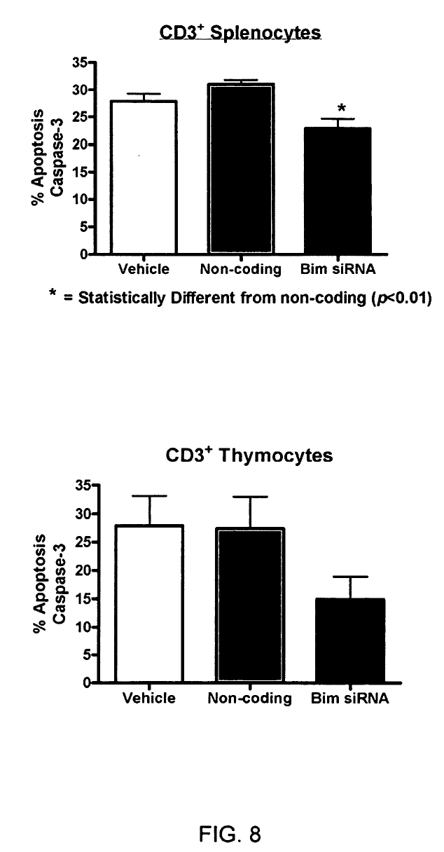

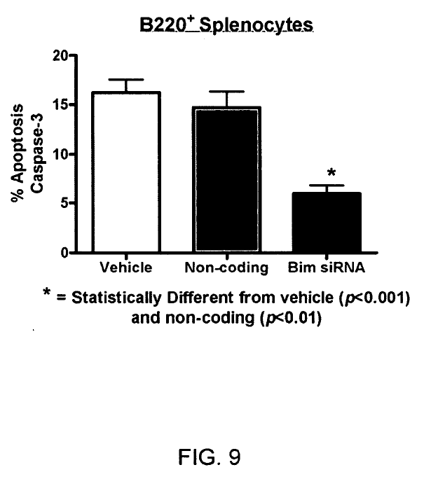

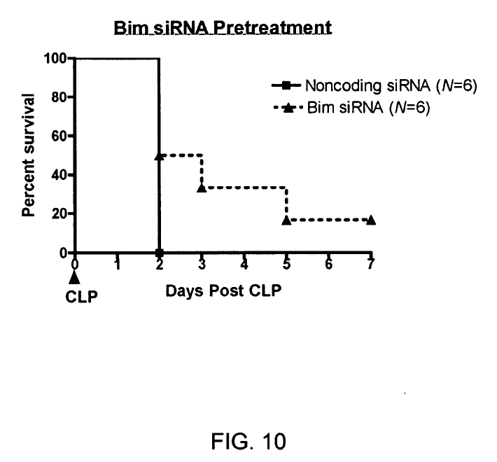

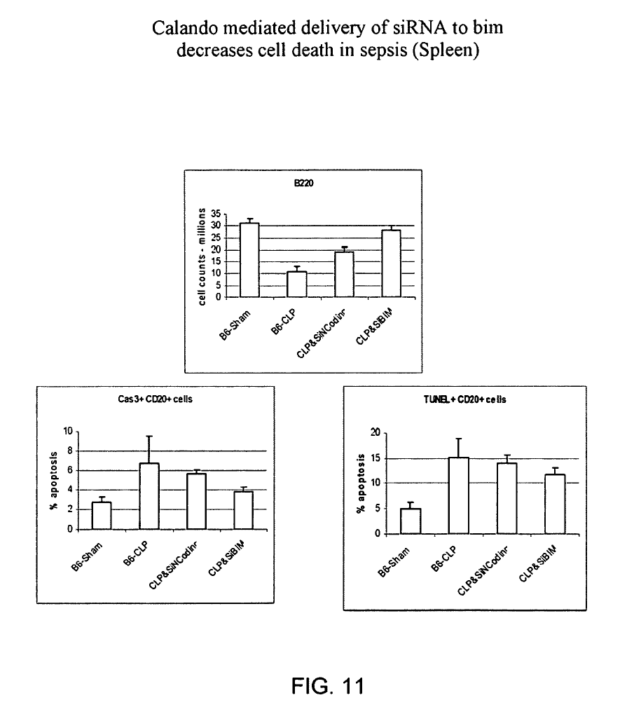

In these experiments, C57BL6 mice (n=29) were treated Kith a single dose of Bim siRNA complexed in cationic liposomes via tail vein injection. 24 hours later mice were subjected to either cecal ligation and puncture (CLP) or sham surgery. Animals were sacrificed at 20 hrs post surgery and spleens were harvested for FACS analysis using TUNEL as a marker for apoptosis. A second cohort of mice (n=30) were followed for survival over 7 days. We observed that the degree of lymphocyte apoptosis in bim siRNA treated mice was markedly decreased compared to negative controls. FACS analysis demonstrated 13.1+/−1.2% B cell apoptosis and 11.5+/−1.5% T cell apoptosis in negative control mice vs. only 2.7+/−0.4% B cell apoptosis and 3.9+/−0.3% T cell apoptosis in bim siRNA treated mice after CLP (p<0.001 and p<0.05, respectively). This striking difference in lymphocyte apoptosis correlated with a significant survival advantage in bim siRNA treated mice. At 7 days there was 17% overall survival in bim siRNA treated CLP mice vs. 0% overall survival in negative control CLP mice (p=0.055). (FIGS. 8, 9 and 10).

### EXAMPLE 15

This example illustrates siRNA against bim decreases spleen cell death in sepsis.

In these experiments siRNA against bim was administered to subject mice immediately after CLP or sham surgery. siRNA against bim was administered with a cyclodextrin based delivery vehicle from Calando (Calando Pharmaceuticals, Inc. Pasadena, Calif.). The amount of apoptosis was determined by measuring active caspase 3 and by TUNEL analysis. As shown in FIG. 11, sepsis is shown to cause a dramatic decrease in absolute cell numbers in spleen and an increase in apoptosis. However, the data show that administration of siRNA against bim prevented cell loss and decreased apoptosis.

### EXAMPLE 16

This example illustrates siRNA against bim decreases thymus cell death in sepsis.

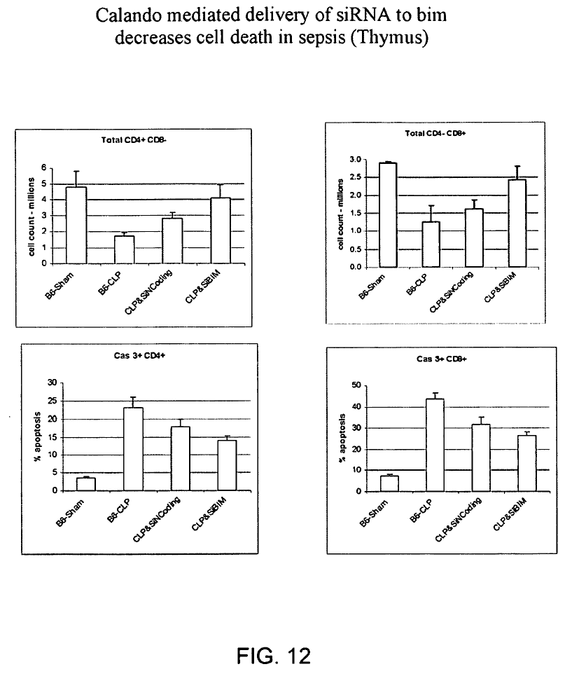

In these experiments, siRNA against bim was administered immediately after CLP or sham surgery. siRNA to bim was administered with a cyclodextrin based delivery vehicle from Calando (Calando Pharmaceuticals, Inc., Pasadena, Calif.). As shown in FIG. 12, Sepsis causes a dramatic decrease in absolute cell numbers in thymus and an increase in apoptosis as determined by active caspase 3. However, administration of the siRNA against bim prevented cell loss and decreased apoptosis.

### EXAMPLE 17

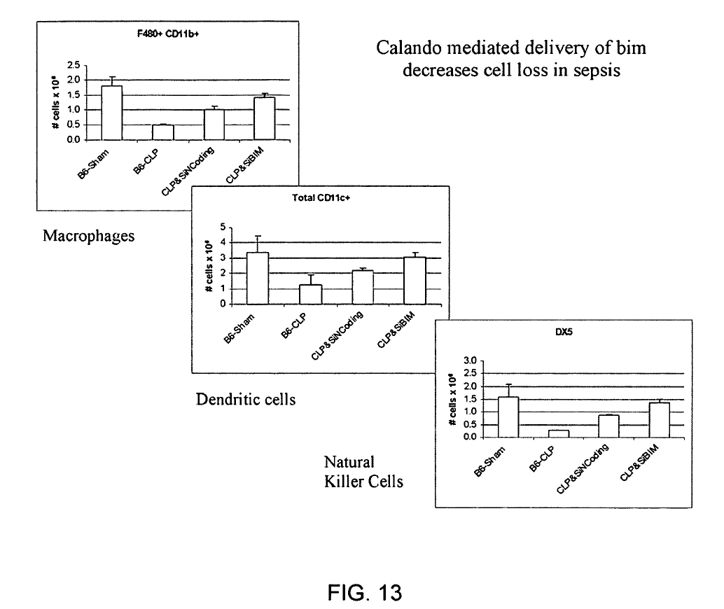

This example illustrates siRNA against bim decreases cell loss in sepsis for macrophages, dendritic cells, and natural killer cells. In these experiments, siRNA against bim was administered immediately after CLP or sham surgery. siRNA against bim was administered with a cyclodextrin based deliver; vehicle from Calando (Calando Pharmaceuticals, Inc., Pasadena, Calif.). As shown in FIG. 13, sepsis causes a dramatic decrease in absolute cell numbers of macrophages, dendritic cells and natural killer cells. However, this sepsis-induced decrease is ameliorated by administration of siRNA against bim.

### EXAMPLE 18

This example illustrates that siRNA against PUMA preserves CD4 T cell numbers in sepsis.

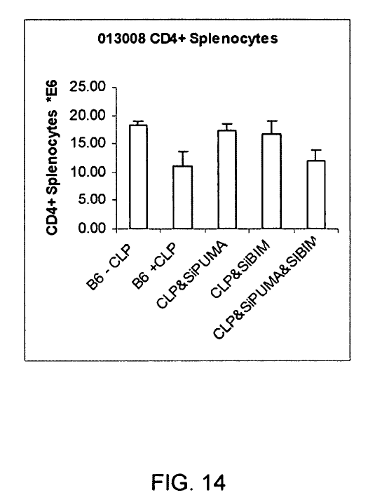

In these experiments, siRNA against PUMA was administered immediately after CLP or sham surgery as in Examples 15-17. Mice were killed 24 hrs later and tissues obtained for absolute cell counts. As shown in FIG. 14, mice that had CLP to induce sepsis and were treated with siRNA against PUMA or Bim had a preservation in their absolute CD4 T cells in spleen compared to mice that did not get the siRNAs.

### EXAMPLE 19

This example illustrates that siRNA against PUMA preserves neutrophil cell numbers in sepsis.

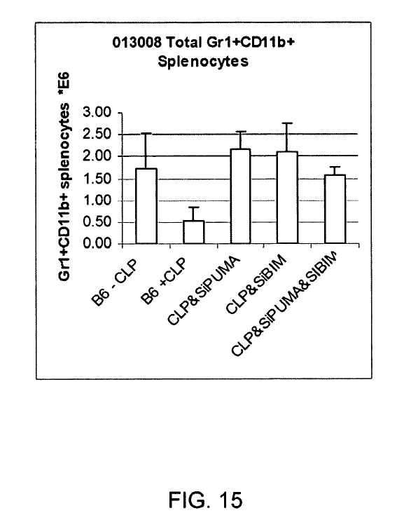

In these experiments, siRNA against PUMA was administered immediately after CLP or sham surgery. Mice were killed 24 hrs later and tissues obtained or absolute cell counts. As shown in FIG. 15, mice that had CLP to induce sepsis and %% ere treated with siRNA to PUMA or Bim had a presentation in their absolute neutrophil counts in spleen compared to mice that did not get the siRNAs.

Examples 20-27 utilize the following materials and methods.

siRNA preparations and delivery

siRNA generation. Target sites for RNA interference were selected using a commercial online program (Dharmacon, http://www.dharmacon.com/sigenome/default.aspx). The 21-23 nucleotide siRNAs were provided in the 2′-deprotected, duplexed, and desalted form of 2′-O-ACE-RNA (Dharmacon). Mouse Bim sequence 5-GGGUGUUUGCAAAUGAUUAUUdTdT-3′ (SEQ ID NO: 6) (sense) and 5′-pUAAUCAUUUGCAAACACCCUUdTdT-3′ (SEQ ID NO: 7) (antisense). Human Bim sequence 5′-UCUUACGACUGUUACGUUAUUdTdT-3′ (SEQ ID NO: 9) (sense) and 5′-pUAACGUAACAGUCGUAAGAUUdTdT-3′ (SEQ ID NO: 10) (antisense). Fluorescence experiments were performed using an identical sequence of Bim siRNA but with a 5-carboxyfluorescein (5-FAM) label incorporated on the 5′ sense strand (Dharmacon). Green fluorescence protein (GFP) experiments were performed using a siRNA to GFP (Dharmacon). GFP sequence 5′-pGGCAAGCACCCUGAAGUUCUUdTdT-3′ (SEQ ID NO: 11) (sense) and 5′-pGAACUUCAGGGUCAGCUUGCCUUdTdT-3′ (SEQ ID NO: 12) (antisense). 21-nucleotide non-targeting siCONTROL siRNA #1 (Dharmacon), containing at least 4 mismatches with all known mouse and human genes as confirmed by BLAST analysis, was used as a control for non-sequence-specific effects.

In vitro siRNA transfection.

Synthetic siRNAs were complexed into cationic liposomes and incubated in vitro as described by Spagnou et al (Biochemistry 43: 13348-13356, 2004). In brief, 5 μl of 1,2-dioleoyl-3-trimethylammonium propane (DOTAP, Roche Diagnostics) was diluted with 20 μl transfection buffer (20 mM HEPES; 150 mM NaCL; pH 7.4). 5 μg siRNA (approximately 0.4 nmol) was diluted with 5 μl transfection buffer. The siRNA solution was transferred into the DOTAP solution, gently mixed, and incubated at 20° C. for 15 minutes. After incubation, the siRNA/DOTAP mixture (20 μl/1 ml well) was added to the reaction well.

In vivo siRNA delivery.

Synthetic siRNAs were complexed into cationic liposomes and delivered via a single tail vein injection as described by Sorensen, D. R., et al., J. Mol. Biol. 327: 761-766, 2003 and Spagnou, S., et al., Biochemistry=43: 13348-13356, 2004. In brief, 50 μl of DOTAP was diluted with 120 μl transfection buffer. 66.5 μg siRNA (approximately 5 nmol) was diluted with 60 μl transfection buffer. The siRNA solution was transferred into the DOTAP solution, gently mixed, and incubated at 20° C. for 15 minutes. After incubation, the siRNA/DOTAP mixture (230 μl/mouse) was injected into mouse tail vein. To dilate the tail veins, mice were warmed under a heat lamp (50° C.) for approximately 3 minutes. The tail vein was punctured using a 27 gauge needle to inject the 230 μl siRNA/DOTAP mixture. A negative control group received 230 μl of DOTAP solution alone.

Human In Vitro Experiments

Determination of Transfection Efficiency.

Fresh whole blood was obtained from healthy human volunteers (n=6). Mononuclear cells were isolated via differential migration over Ficoll-Paque Plus® and counted on a ViCell A automated cell counter (Beckman Coulter). 2×10⁶ cells were plated in 1 ml very low endotoxin medium RPMI 1640 and treated with 5-carboxyfluorescein (5-FAM) labeled Bim-targeting siRNA/DOTAP mixture (5 μg/5 μg), or with unlabeled Bim-targeting siRNA/DOTAP mixture (5 μg/5 μg). After 4 hours of incubation at 37° C., 10% fetal bovine serum was added and all samples were then incubated for an additional 16 hours at 37° C. After incubation, mononuclear cells were harvested for flow cytometric analysis. Flow cytometric analysis of mean fluorescence intensity (50,000 events/sample) was performed on FACScan (BD Biosciences).

Detection and Quantification of Apoptosis.

Human mononuclear cells were obtained from healthy human volunteers (n=6) as described. 2×10⁶ cells were plated in 1 ml very low endotoxin medium RPMI 1640 and treated with Bim-targeting siRNA/DOTAP mixture (5 μg/5 μg), non-targeting siRNA/DOTAP mixture (5 μg/5 μg), or DOTAP alone (5 μg). After 4 hours of incubation at 37° C. experimental samples were irradiated with 15Gy γ-irradiation (Co60 source, J. L. Shepard and Associates). Control samples received no irradiation. 10% fetal bovine serum was added to all samples which were then incubated for an additional 16 hours at 37° C. After incubation, mononuclear cells were harvested for flow cytometric analysis. Apoptosis was quantified using phycoerythrin-labeled terminal deoxynucleotidyl transferase-mediated dUTP-biotin nick end-labeling (TUNEL, Phoenix Flow Systems, Inc.) or by staining for active caspase 3 with a primary rabbit anti-mouse caspase 3 antibody and a phycoerythrin-labeled donkeys anti-rabbit secondary antibody (Cell Signaling Technology) as previously described (Hotchkiss, R. S., et al., J. Immunol. 174: 5110-5118, 2005; Bommhardt, U., et al., J. Immunol. 172: 7583-7591, 2004, Schwulst. S. J., J. Immunol. 177: 557-565, 2006). Human T cells were identified using a fluorescein (FITC)-labeled anti-human CD3 antibody (BD Pharmingen). Flow cytometric analysis (50,000 events/sample) was performed on FACScan (BD Biosciences). All human studies were approved by the Washington University Institutional Review Board.

Murine In Vivo Experiments

Animals. Male C57BL/6-TgN(ACTbEGFP)1 Osb mice 7-9 weeks old weighing approximately 20-25 grams that express green fluorescent protein (GFP) using a β-actin promoter were used to evaluate tissue specificity of in vivo siRNA deliver, (The Jackson Laboratories). Mice used in acute interferon activation studies, apoptosis studies, gene knockdown studies, and survival studies were C57BL/6 male mice 7-9 weeks old weighing approximately 20-25 grams (The Jackson Laboratories).

Sepsis Model.

The cecal ligation and puncture (CLP) technique as developed by Chaudry et al. (Surgery. 85: 205-211, 1979) and modified by our laboratory (Muenzer, J. T., et al., Shock. 26: 565-570, 2006) was used to induce septic peritonitis. Briefly, mice were anesthetized using 2% halothane with supplemental oxygen. A 1 cm left paramedian incision was made and the cecum %% as ligated below the ileocecal valve. The cecum was punctured once with a 27 gauge needle. Sham mice had cecal manipulation only. The incision was sutured with 4-0 silk suture. Mice were given 1 ml of 0.9% saline subcutaneously immediately postoperatively. They %% ere then allowed free access to food and water. Animal studies were approved by the Washington University Animal Studies Committee.

Tissue Distribution and Functionality of siRNA.

GFP transgenic mice were injected with either 5 nmol GFP siRNA complexed to 50 μg DOTAP, 5 nmol non-coding siRNA complexed to 50 μg DOTAP, or vehicle alone. Multiple tissue types were examined 24 hours post-injection using both fluorescence microscopy and flow cytometry. Freshly isolated organ tissue-sections were obtained using a microtome and examined as wet tissue sections using a Nikon Eclipse E600 microscope with a FITC filter (Nikon USA, El Segundo, Calif.) at 200× magnification. Identical illumination and acquisition settings were used for all samples to enable an accurate assessment of the “knockdovnm” effects of the GFP siRNA. The images were not processed by any image analysis software.

Assessment of IL-6 and IFN-γ activation. Naïve and CLP mice were injected with 230 μl of saline (n=4), vehicle (n=4), DOTAP-complexed Bim siRNA (n=4), or DOTAP-complexed non-targeting siRNA (n=4) via a single tail vein injection. Mice were sacrificed and plasma was harvested 20 hours post-injection. The circulating levels of IL-6 and IFN-γ was analyzed in triplicate using an enzyme-linked immunosorbent assay (ELISA) kit (San Jose, Calif., BD Biosciences,) according to the manufacturer's recommendations.

Assessment of IFN-α, and IFN-β.

Naïve mice were injected with 230 μl of saline (n=2), vehicle (n=2). DOTAP-complexed Bim siRNA (n=2), or DOTAP-complexed non-targeting siRNA (n=2), via a single tail vein injection. A fifth group underwent CLP (n=2) as a positive control. Mice were sacrificed and plasma was harvested 8 hours post-injection. The circulating levels of IFN-α and IFN-β were analyzed in duplicate using an enzyme-linked immunosorbent assay (ELISA) kit (PBL Biomedical Laboratories, Piscataway, N.J.) according to the manufacturer's recommendations.

Determination of protein expression. Flow cytometry. Mice were injected with 230 μl of either DOTAP-complexed Bim siRNA or DOTAP-complexed non-targeting siRNA via a single tail vein injection 24 hours prior to CLP. Mice were then sacrificed 24 hours post-injury and spleens and thymi were obtained. Whole splenocytes and thvmocytes were isolated and Bim expression was quantified using a primary rabbit anti-mouse Bim antibody and a fluorescein (FITC)-labeled donkey anti-rabbit secondary antibody (Cell Signaling Technology). Similarly, Bcl-2 expression was quantified using a phycoerythrin-labeled hamster anti-mouse Bcl-2 antibody (BD Pharmingen). Mouse B- and T-cells were identified using cyanine 5 (Cy5)-labeled anti-mouse B220 and anti-mouse CD3 Abs, respectively (BD Pharmingen). Three channel flow cytometric analysis (50,000 events/sample) was performed on FACScan (BD Biosciences) as previously described (Hotchkiss, R. S., et al., J. Immunol. 174: 5110-5118, 2005; Bommhardt, U., et al., J. Immunol. 172: 7583-7591, 2004; Schwulst, S. J., J. Immunol. 177: 557-565, 2006).

Detection and Quantification of Apoptosis.

Naive mice were injected with 230 μl of either Bim siRNA/DOTAP mixture, non-targeting siRNA/DOTAP mixture, or DOTAP mixture alone via a single tail vein injection. 24 hours post injection mice underwent CLP (n=20) or sham (n=9) surgery as described above. 20 hours post-injury mice were sacrificed and spleens and thymi were obtained. Whole splenocytes and thymocytes were isolated and apoptosis was quantified using TUNEL or intracellular staining for active caspase-3 as described above. Mouse B and T cells were identified using fluorescein (FITC)-labeled anti-mouse B220 and cyanine 5 (Cy5)-labeled anti-mouse CD3 Abs, respectively (BD Pharmingen). Flow cytometric analysis (50,000 events/sample) was performed on FACScan (BD Biosciences) as previously described (Hotchkiss, R. S., et al., J. Immunol. 174: 5110-5118, 2005; Bommhardt, U., et al., J. Immunol. 172: 7583-7591, 2004; Schwulst, S. J., J. Immunol. 177: 557-565, 2006).

Survival. Mice were injected with 230 μl of either Bim-targeted siRNA/DOTAP mixture (n=16) or non-targeting siRNA/DOTAP mixture (n=16) via a single tail vein injection 24 hours prior to CLP, the day of CLP, and the day following CLP (3 total injections). Survival was recorded for seven days.

Statistical Analysis

Data reported are the mean±SEM. Data were analyzed with the statistical software program PRISM (GraphPad Software. San Diego, Calif.). Data involving two groups were analyzed by a student's t test, while data involving more than two groups were analyzed using one-way

### EXAMPLE 20

This example illustrates high transfection efficiency of siRNA in human cells by liposome delivery

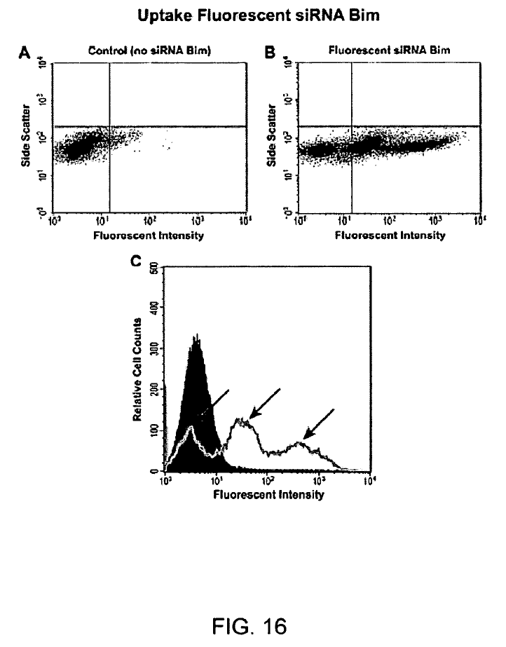

In these experiments, the characteristic forward and side scatter properties of lymphocytes were used to identify the lymphocyte gate as previously described (Hotchkiss, R. S., et al., J. Immunol. 174: 5110-5118, 2005). Back-gating of surface-marked lymphocytes (CD3+) was used to confirm the appropriate lymphocyte gate. In order to determine the efficiency of transfection of our siRNAs with DOTAP, we utilized an in vitro system in which human peripheral blood lymphocytes were examined for mean fluorescence intensity via flow cytometry using a 5-FAM labeled siRNA. Lymphocytes transfected with the 5-FAM labeled siRNA demonstrated an average eight-fold increase in mean fluorescence intensity as compared to nonlabeled controls (p<0.0001; FIG. 16A,B). Interestingly, we observed three distinct cell populations, one corresponding to nontransfected cells and two cell populations with different degrees of siRNA uptake (FIG. 16C). This phenomenon has been previously described and likely corresponds to varying degrees of transfection efficiency with the highest peak corresponding to greater uptake of labeled siRNA molecules (Martinez-Ferrandis, J. I., et al., Cytometry A 71: 599-604, 2007).

### EXAMPLE 21

This example illustrates that Bim-targeted siRNA protects human peripheral blood lymphocytes from radiation-induced apoptosis.

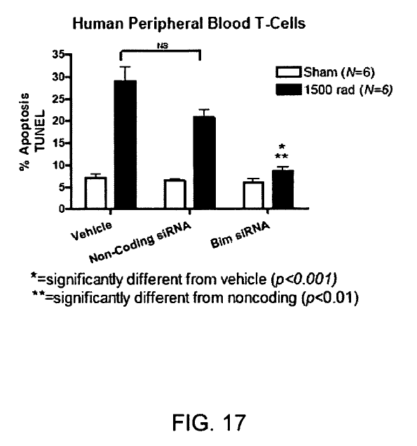

In order to examine whether Bim-targeted siRNA protects against apoptosis, we utilized an in vitro system in which human peripheral blood mononuclear cells were transfected with Bim-targeted siRNA and injured with γ-irradiation. Human lymphocytes were identified as described above and examined for apoptosis via flow cytometry. TUNEL labeling demonstrated marked protection against γ-irradiation-induced apoptosis in CD3+ peripheral blood lymphocytes (p<0.001) (FIG. 17).

### EXAMPLE 22

This example illustrates tissue distribution and functionality of siRNA after low volume liposome mediated delivery.

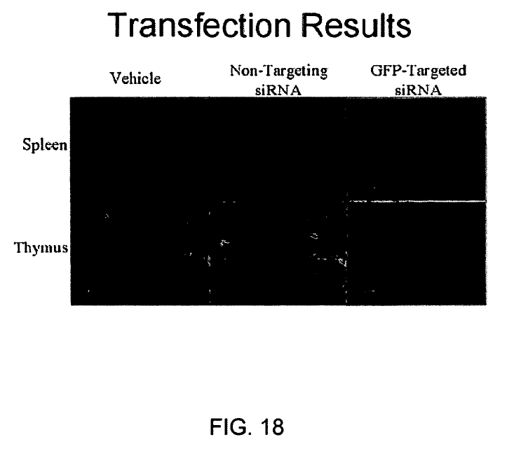

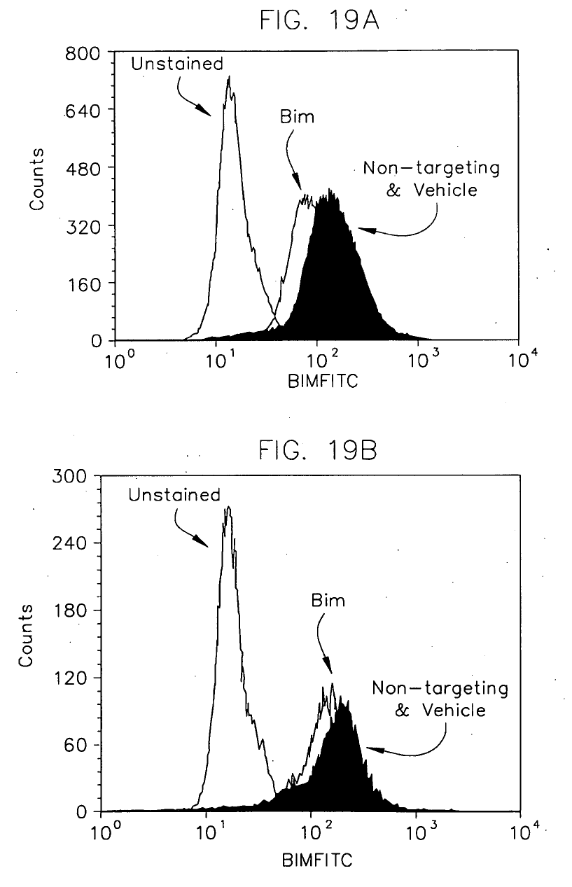

The in vivo use of liposome delivered siRNA is relatively new (Kim, S. I., et al., Mol. Ther. 15: 1145-1152 2007; Hassan, A., et al, Physiol Genomics. 21: 382-388, 2005; Sioud, M., et al., Biochem Biophys Res Commun. 312:1220-1225, 2003.). Therefore, as described by the laboratory of Ayala (Wesche-Soldato, D. E., et al., Blood 106: 2295-2301, 2005), we initially sought to determine both the capacity of siRNA to suppress specific GFP transgene expression as well as to define the tissue distribution of siRNA when delivered via low volume liposome-complexed tail vein injection. To answer these questions, GFP transgenic mice were injected with either 5 nmol GFP siRNA complexed to 50 μg DOTAP, 5 nmol non-coding siRNA complexed to 50 μg DOTAP, or vehicle alone as described in the material and methods section. Animals treated with the GFP-targeted siRNA demonstrated a decrease in fluorescence in spleen and thymus, (FIG. 18). However, no change was observed in the heart, lung, and liver (data not shown). These changes in fluorescence were confirmed via flow cytometry by direct quantitation of mean fluorescence intensity (data not shown). These data indicate that low volume lipsome-complexed siRNA suppressed the GFP transgene in some, but not all, fissue types.

### EXAMPLE 23

This example illustrates that systemic delivery of liposome-complexed siRNA does not alter cytokine secretion in naïve animals or animals subjected to cecal ligation and puncture (CLP).

In these experiments, serum samples from mice injected with liposome-complexed siRNA against Bim or non-targeting siRNA, liposomes alone, or saline alone were measured for levels of IFN-γ and IL-6. Levels of IL-6 and IFN-γ did not differ significantly between animals treated with Bim siRNA, non-targeting siRNA, liposomes alone, or saline alone. The serum abundance of both IFN-β and IL-6 were not detectable in the plasma of any naive animal (n=4 per group). CLP animals had mild increases in both IL-6 and IFN-γ at 20 hours but did not significantly differ between animals treated with Bim siRNA, non-targeting siRNA, liposomes alone, or saline alone.

### EXAMPLE 24

This example illustrates that systemic delivery of liposome-complexed siRNA causes a mild increase in IFN-α and IFN-β.

DOTAP-complexed siRNA has been used in vitro for several years, there has been some evidence that its use in vivo results in a potent induction of interferon responses (Ma, Z., et al., Biochem Biophys Res Commun. 330: 755-759, 2005). In these experiments, serum from CLP mice or mice injected with liposome-complexed siRNA against Bim or non-targeting siRNA, liposomes alone, or saline alone were analyzed for circulating levels of IFN-α, IFN-β. Levels of IFN-α and IFN-β were mildly elevated in animals receiving liposome-complexed siRNA against Bim or non-targeting siRNA (50-100 pcg/ul) as compared to the other groups (5-25 pcg) (data not shown).

### EXAMPLE 25

This example illustrates that low volume liposome-complexed delivery of Bim siRNA decreases Bim protein expression in septic mice.

Sepsis induces alterations in the expression of hundreds of genes including a number of bcl-2-related apoptosis genes such as bim (Wagner, T. H., et al., Am. J. Physiol. Regul. Integr. Comp. Physiol. 292:1751-1759, 2007). In these experiments, flow cytometry analysis demonstrated a 33.1±2.9% decrease in Bim protein expression in the B cells of septic mice (FIG. 19A, p<0.01) and a 19.5±2.5% decrease in Bim protein expression in the T cells of septic mice (FIG. 19B, p<0.01), as determined by the change in mean fluorescence intensity. There was no effect of non-targeting siRNA or vehicle on Bim protein expression.

### EXAMPLE 26

This example illustrates that Bim siRNA blocks sepsis-induced lymphocyte apoptosis in spleen and thymus.

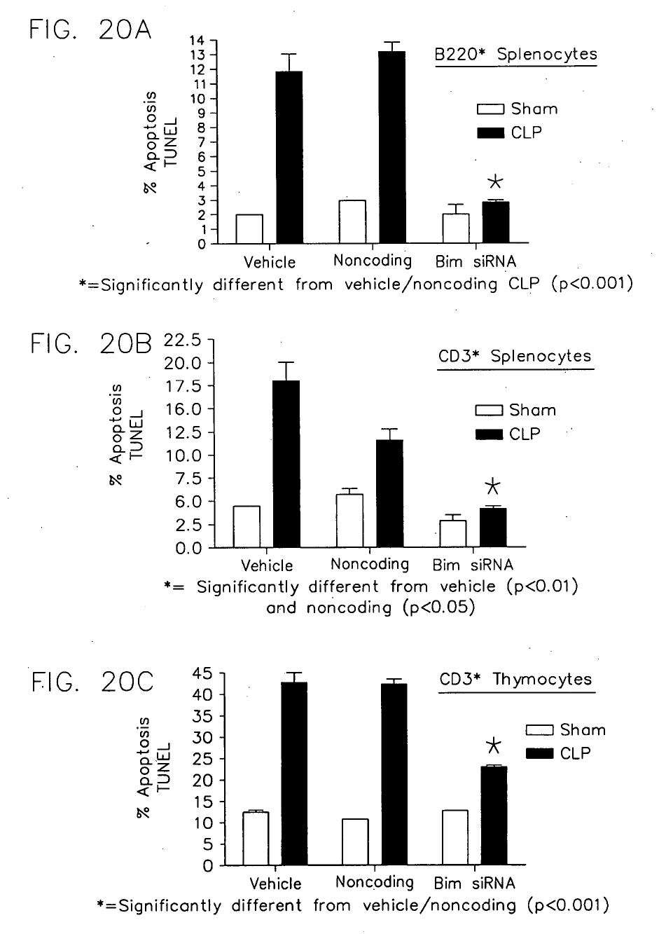

Sepsis-related immune dysfunction is, in part, driven by a profound apoptosis-induced loss of lymphocytes. Numerous studies have demonstrated that prevention of this sepsis-induced lymphocyte apoptosis improves survival in various animal models of sepsis (Hotchkiss, R. S., et al., Proc. Natl. Acad. Sci. USA 96: 14541-14546, 1999; Hotchkiss, R. S., et al., Nat. Immunol. 1: 496-501, 2000; Bommhardt, U., et al., J. Immunol. 172: 7583-7591, 2004; Schwulst, S. J., et al., J. Immunol. 177: 557-565, 2006). Therefore, we determined whether siRNA against Bim could prevent the onset of sepsis-induced apoptosis. CLP caused a marked increase in both splenic B cell and splenic T cell apoptosis as compared to sham (FIG. 20). This increase in splenic B cell apoptosis was ameliorated by treatment with Bim-targeted siRNA as demonstrated by both TUNEL (p<0.001; FIG. 20A) and active caspase 3 labeling (p<0.001; data not shown). Likewise, splenic T cell apoptosis was reduced to sham levels after treatment with Bim-targeted siRNA as demonstrated by both TUNEL (p<0.01; FIG. 20B) and active caspase-3 labeling (p<0.01; data not shown). Additionally, CLP caused a massive increase in thymocyte apoptosis and, although not completely protective, treatment with Bim-targeted siRNA significantly reduced the degree of sepsis-induced thymocyte apoptosis as demonstrated by both TUNEL (p<0.001; FIG. 5C) and active caspase-3 labeling (p<0.001; data not shown).

### EXAMPLE 27

This example illustrates that treatment with Bim siRNA improves survival in a murine model of septic peritonitis.

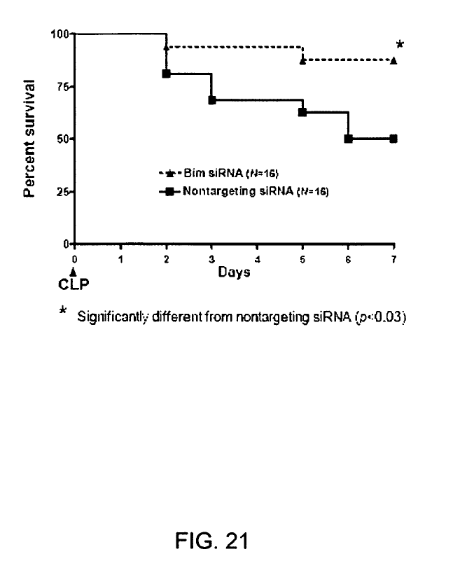

Our laboratory has recently shown that Bim null mice have a marked survival advantage after CLP as compared to wild type controls (Chang, C. K. et al., FASEB J. 21:708-719, 2007.). Therefore, we aimed to determine whether functional Bim knockdown using Bim siRNA could reproduce the survival advantage as seen in Bim null mice. Mice were pretreated with either 5 nmol of liposome-complexed Bim siRNA or non-targeting siRNA 24 hours prior to injury, on the day of injury, and 24 hours post injury. Survival was recorded for 7 days. Mice receiving Bim siRNA had a 90% survival while those receiving non-targeting siRNA had a survival of only 50% at 7 days (p<0.03; FIG. 21).

### EXAMPLE 28

This example illustrates that siRNA against bax and bak reduces apoptotic cell death in septic animals.

In these experiments, C57BL/6 mice were pretreated by tail vein injection with siRNA to Bax and Bak or non-coding control in DOTAP transfection agent. The sequences of the siRNAs were, for mouse Bak sense strand—CGACACAGAGUUCCAGAAUUU (SEQ ID NO: 13), and for mouse Bax sense strand GAGAUGAACUGGACAGCAAUU (SEQ ID NO: 14). Mice received cecal ligation and puncture (CLP) 24 hrs after the initial injection and another siRNA treatment 3-4 hours post-injury. 24 hours post-injury, mice were sacrificed. Splenocytes and thymocytes were harvested at the time of sacrifice from all mice for histology and flow cytometry. Apoptosis was quantified using a commercially available antibody against active caspase-3 and Tunel. Mouse T and B cell populations were identified using fluorescein-labeled anti-mouse CD3 and fluorescein-labeled anti-mouse b220 antibodies, respectively. These methods were used in Examples 24-27.

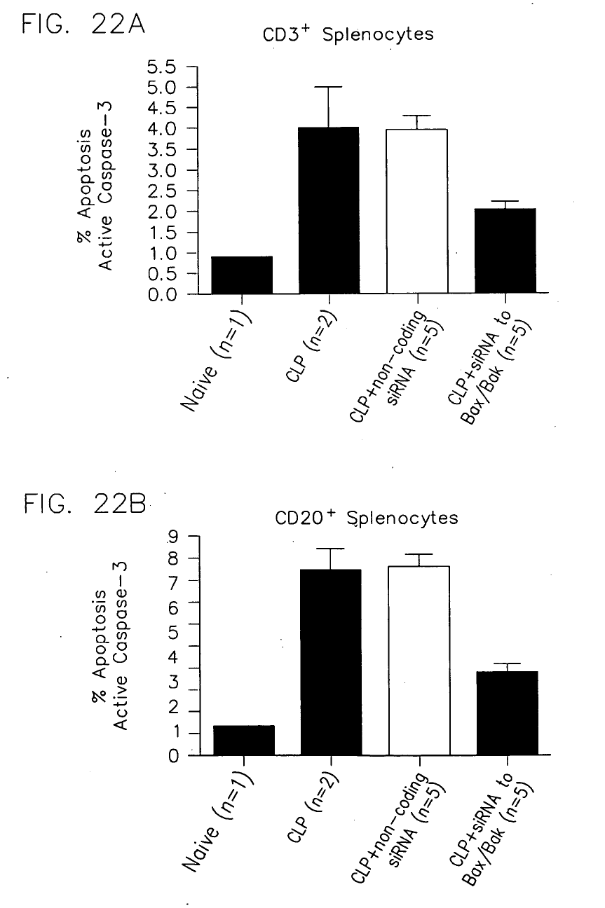

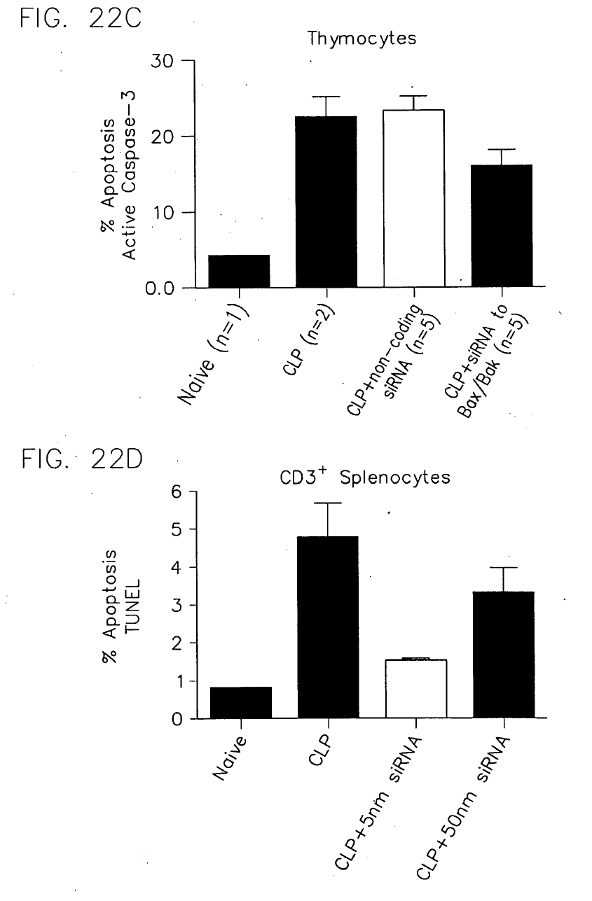

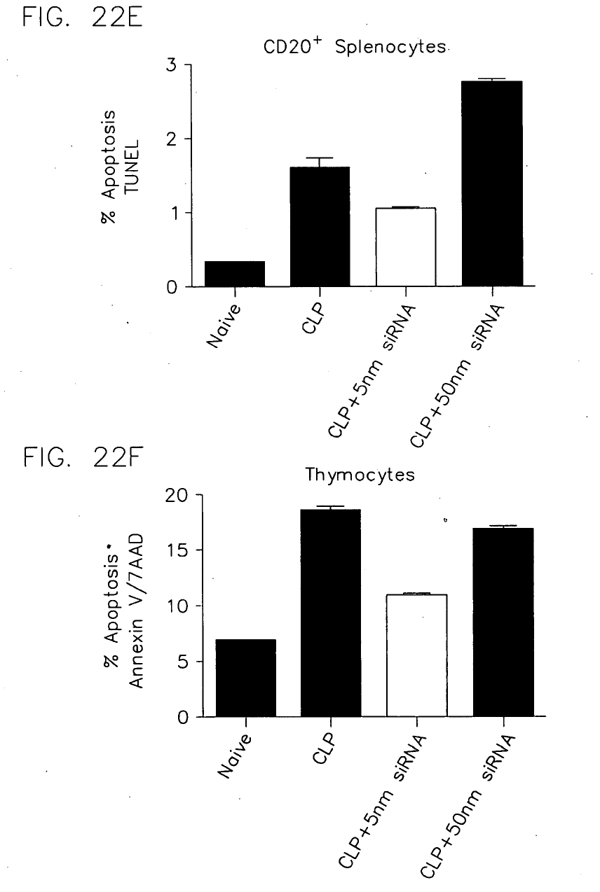

Flow cytometry analysis (FIG. 22) revealed a reduction in apoptotic cell death in all cell types tested from animals receiving siRNA to Bax and Bak, in comparison to cells from control animals receiving non-coding siRNA or no therapy.

### EXAMPLE 29

This example illustrates uptake of fluorescent siRNA by electroporation.

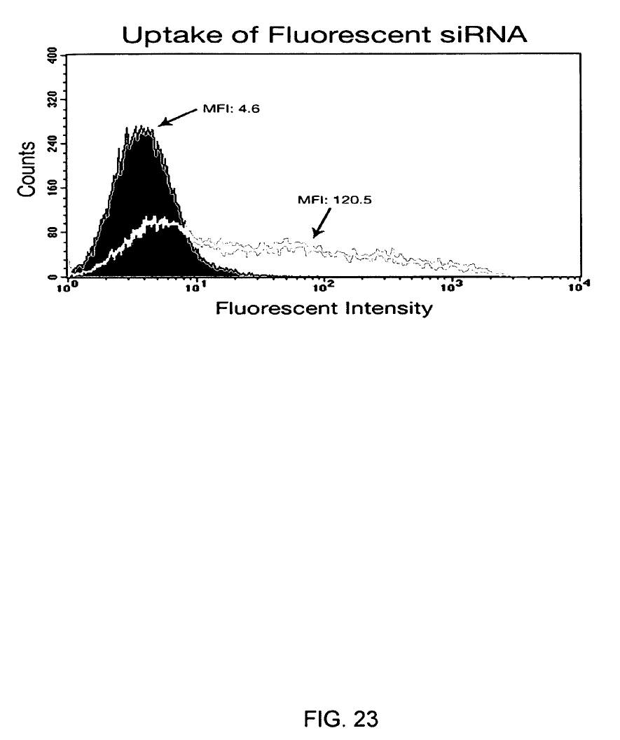

In these experiments, mouse splenocytes were suspended in transfection buffer at 10×10⁶ cells/ml and electroporated in 0.4-cm cuvettes with 25 mg siRNA at a voltage of 250 mV and a capacitance of 250 mF As shown in FIG. 23, cells transfected with fluorescently-labeled siRNA via electroporation showed significantly greater levels of fluorescence compared to controls, measured by mean fluorescent intensity (MFI), demonstrating successful uptake of the siRNA.

### EXAMPLE 30

This example illustrates that siRNA against bax and bak protects cells from apoptotic cell death in vitro.

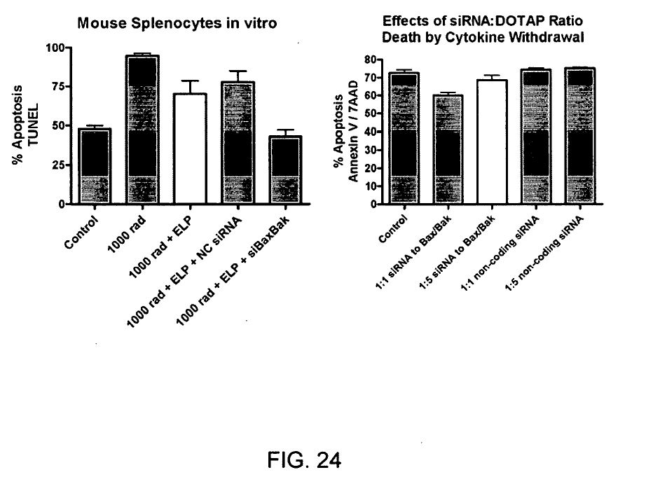

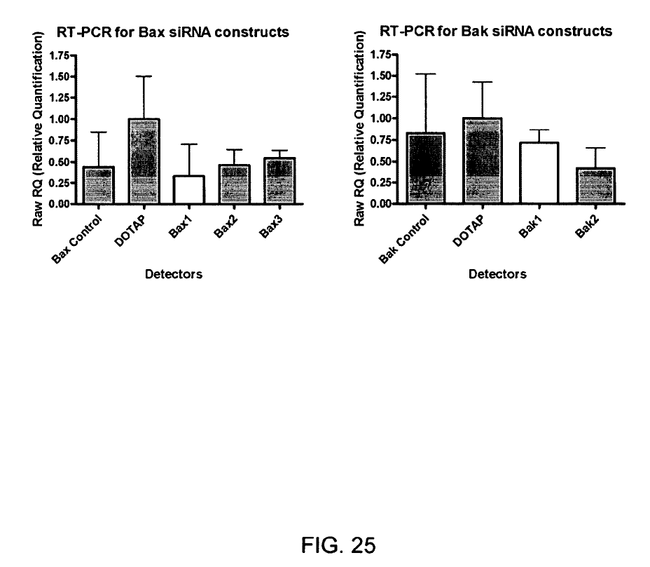

In these experiments, splenocytes were harvested from C57BL/6 mice, with or without CLP. Lymphocytes were isolated and transfected with siRNA via electroporation or cationic lipid transfection using DOTAP as the transfection agent. Cells were exposed to radiation 3-6 hrs post-transfection or allowed to die by cytokine withdrawal. At 24 hrs, cells were harvested and apoptosis was quantified using flow cytometry. As shown in FIG. 24 left panel, irradiated mouse splenocytes receiving siRNA against bax and bak showed less apoptosis than cells receiving a control siRNA. The right panel shows the effects of various ratios of siRNA to DOTAP on cell death levels following cytokine withdrawal. Protection could be seen in cells receiving siRNA to Bax and Bak, but cell viabilities and absolute cell counts showed no differences among groups receiving coding siRNA, non-coding siRNA, and no siRNA therapy (data not shown). Toxic effects of electroporation and cationic lipids were also seen.

### EXAMPLE 31

This example illustrates upregulation of Bax and Bak in cells receiving only cationic transfection agent, while cells receiving siRNA to Bax and Bak show Bax and Bac mRNA levels comparable to or lower than mRNA levels in control cells. In these experiments, Splenocytes were harvested from C57BL/6 mice and lymphocytes were isolated and transfected with siRNA via cationic lipid transfection (DOTAP). mRNA was isolated from primary lymphocytes using a commercially available RNA isolation kit A cDNA library was constructed and mRNA levels were quantified by means of real-time PCR (RT-PCR). Results (FIG. 25) show expression levels for Bax (left panel) and Bak (right panel).

### EXAMPLE 32

This example illustrates methods of selecting, designing, and using RNAi, siRNA, shRNA, and other ribonucleic acids.

The present teachings disclose methods using siRNA. Many U.S. patent and patent applications provide methods for preparing and using siRNA, including but not limited to, those with application Ser. Nos. 09/821,832, 10/490,955, 10/255,568, 10/832,248, 10/433,050, 10/832,432, 10/832,257, 11/142,865, 11/142,866, 11/474,738, 11/474,919, 11/474,930, 11/474,932, 10/349,320, 10/384,260, 10/382,634, 10/382,768, 10/382,395, 09/866,557, 10/055,797, 09/858,862, 10/350,798, 10/997,086, 11/330,043, 10/759,841, 10/646,070, 10/821,710, 11/218,999, 11/180,928, 10/821,726, 10/346,853, 09/997,905, 09/100,813, 09/646,807, 09/100,812, 90/007,247, 90/008,096, 09/215,257, 10/283,267, 10/283,190 and 10/282,996 (all of which are herein incorporated by reference).

### EXAMPLE 33

This example illustrates protection from cell death in sepsis using an siRNA against Fas-associated death domain (FADD).

Our previous work showed that mice deficient in Fas associated death domain (FADD) are protected from cell death in sepsis (Chang, K. C., et al., FASEB J. 21: 708-719, 2007). Accordingly, an siRNA against FADD is constructed using methods well known to skilled artisans, and mixed with DOTAP to form a complex. The complex is then administered to a subject in sepsis, and apoptosis is reduced.

### EXAMPLE 34

This example sets forth examples of genes for which siRNA can be produced and used to prevent apoptosis in sepsis in accordance with the present teachings.

Genes that will function in the present teachings include but are not limited to those listed in the Table directly below. The inventors have shown that mice which lack these genes have reduced apoptosis in sepsis. For each gene the table lists the EntrezGene Gene ID (updated Jan. 29, 2007). The EntrezGene entry including (1) alternative nomenclature for each gene, (2) full sequence information for the genomic and transcribed sequence of the gene as well as information regarding transcriptional variants, (3) known genetic variation within the sequence of the full length gene including 3′-UTR. 5′-UTR, all introns and exons, (4) related sequences (mRNA, genomic and protein sequences), all of which are incorporated herein as are all other elements in each GeneID.

| Gene Name | EntrezGene GeneID |
|---|---|
| Bim | 10018 |
| Bid | 637 |
| Bad | 572 |
| PUMA | 27113 |
| NOXA | 5366 |

siRNA Design:

Methods to design functional siRNA take into account the following: (1) sequence, structure and self-homology of the siRNA with special attention paid to the nucleotides at the 3′- and 5′-termini of the siRNA, (2) sequence and structure of the targeted region of the mRNA, (3) the energetics of the duplex formed between the siRNA and its target mRNA, (4) interactions between the siRNA and RNA-processing enzymes and macromolecular complexes—for example RNAses, Dicer, RISC, toll-like receptors, MDA5, (5) interactions between the siRNA/target mRNA duplex and double-stranded RNA processing enzymes and macromolecular complexes.

siRNA sequences can be selected using one or more of (1) manual or automated implementation of algorithms that incorporate one or more of the above parameters into the selection of siRNA sequences, (2) exhaustive or representative library screening, (3) genomic sequences from pathogenic organisms. siRNA sequences can be further optimized using non-natural nucleosides or non-natural backbones. siRNA sequences do not have to be fully complementary to the targeted mRNA sequence, substitutions are permitted at numerous sites (for example, see PLoS Genetics; Schwarz D S, Ding H, Kennington L, Moore J T, Schelter J, et al. (2006) Designing siRNA That Distinguish between Genes That Differ by a Single Nucleotide. PLoS Genet. 2(9): e140).

All articles and references referred to via PMID in this example are herein incorporated by reference in there entirety.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

All publications, patents, patent applications and other references cited in this application are herein incorporated by reference in their entirety as if each individual publication, patent, patent application or other reference were specifically and individually indicated to be incorporated by reference. Applicants reserve the right to challenge assertions and conclusions set forth by the authors of any reference cited herein.

It is to be further understood that the embodiments set forth in the present teachings are not intended as being exhaustive or limiting of the invention, and that many alternatives, modifications, and variations will be apparent to those skilled in the art in light of the foregoing examples and detailed description. Accordingly, the present specification is intended to embrace all such alternatives, modifications, and variations that fall within the spirit and scope of the following claims.

What is claimed is:

1. A method of treating sepsis, comprising administering to a subject in need of treatment of sepsis a therapeutically effective amount of a composition comprising at least one siRNA directed against at least one gene encoding a pro-apoptotic polypeptide other than Fas or caspase-8.

2. A method of treating sepsis in accordance with claim 1, wherein the at least one siRNA is directed against at least one pro-apoptotic component of a mitochondrial apoptosis pathway.

3. A method of treating sepsis in accordance with claim 2, wherein the at least one pro-apoptotic component of a mitochondrial apoptosis pathway is at least one pro-apoptotic Bcl-2 family member.

4. A method of treating sepsis in accordance with claim 3, wherein the at least one pro-apoptotic Bcl-2 family member is a BH3-only protein.

5. A method of treating sepsis in accordance with claim 3, wherein the at least one pro-apoptotic Bcl-2 family member is bim.

6. A method of treating sepsis in accordance with claim 3, wherein the at least one pro-apoptotic Bcl-2 family member is puma.

7. A method of treating sepsis in accordance with claim 3, wherein the composition comprises a first siRNA directed against bax and a second siRNA directed against bak.

8. A method of treating sepsis in accordance with claim 1, wherein the composition further comprises a cyclodextrin.

9. A method of treating sepsis in accordance with claim 8, wherein the cyclodextrin is a linear, cyclodextrin-containing polycation.

10. A method of treating sepsis in accordance with claim 9, wherein the composition further comprises a poly(ethylene glycol) (PEG).

11. A method of treating sepsis in accordance with claim 10, wherein the PEG is a PEG comprising a terminal adamantane.

12. A method of treating sepsis in accordance with claim 11, wherein the composition comprises a plurality of nanoparticles comprising the PEG, the cyclodextrin and the at least one siRNA.

13. A method of treating sepsis in accordance with claim 1, wherein the composition further comprises a cationic lipid.

14. A method of treating sepsis in accordance with claim 13, wherein the cationic lipid is 1,2-dioleoyl-3-trimethylammonium propane (DOTAP).

15. A method of inhibiting apoptosis in a subject, comprising administering to a subject in need of treatment of sepsis, a composition comprising at least one siRNA directed against at least one gene encoding a pro-apoptotic polypeptide other than Fas or caspase-8, in an amount effective for inhibiting apoptosis in at least one cell type that exhibits increased apoptosis in sepsis.

16. A method of inhibiting apoptosis in a subject in accordance with claim 15, wherein the at least one cell type that exhibits increased apoptosis in sepsis is selected from the group consisting of lymphocytes, dendritic cells, macrophages/monocytes and natural killer cells.

17. A method of inhibiting apoptosis in a subject in accordance with claim 15, wherein the at least one cell type that exhibits increased apoptosis in sepsis is selected from the group consisting of splenocytes and thymocytes.

18. A method of inhibiting apoptosis in a subject, comprising administering to a subject in need of treatment of sepsis, a composition comprising at least one siRNA directed against at least one gene encoding a Fas associated death domain (FADD), in an amount effective for inhibiting apoptosis in a cell type that exhibits increased apoptosis in sepsis.
